(19) United States
(12) Patent Application Publication
(10) Pub. No.: US 2009/0318454 A1
(43) Pub. Date: Dec. 24, 2009

(54) NOVEL ANTIOXIDANTS AND METHODS OF TREATMENT

(76) Inventors: Carl P. Weiner, Mission Hills, KS (US); Peter Swaan, Laurel, MD (US); Jeffrey Aube, Lawrence, KS (US); Abhijit Ray, Salt Lake City, UT (US)

Correspondence Address: Workman Nydegger;1000 Eagle Gate Tower, 60 East South Temple, Salt Lake City, UT, 84111, US

(21) Appl. No.: 12/410,133

(22) Filed: Mar. 24, 2009

Related U.S. Application Data
(60) Provisional application No. 61/070,555, filed on Mar. 24, 2008.

(51) Int. Cl. A61K 31/53; C07C 251/60; A61K 31/195; C07D 253/065

(52) U.S. Cl. 514/242; 562/440; 562/426; 514/562; 514/567; 544/182

(57) ABSTRACT

Formula 1 is an antioxidant compound, wherein R, R1, or D is an antioxidant substituent; R and D are each independently selected from hydrogen or groups containing cysteine, thiols, disulfides, amino acids, amines, amides, or carboxylic acids; when one of R or D is hydrogen, the other includes a thiol, disulfide, or carboxylic acid; A is CO, SO, SO₂, or C═S; E is one or more ring groups which are aromatic, carbocyclic, and/or heterocyclic 5, 6 and 7 membered rings being mono, bi, tri, tetra, penta, hexa, hepta or octa cyclic fused rings that are substituted or unsubstituted, each heterocyclic ring can include hetero atoms chosen from to O, S, N, Se, or P; each R1 is at a para, meta, and/or ortho position; n is 1, 2, 3, 4, or 5 for each ring; and each R1 is hydrogen or an antioxidant substituent.

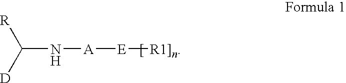

### CROSS-REFERENCE TO RELATED APPLICATIONS

The present patent application claims benefit of U.S. Provisional Application Ser. No. 60/070,555, entitled “SPIN TRAPPING GLUTATHIONE PRECURSOR/PROMOIETY: A POWERFUL ANTIOXIDANT WITH DUAL MECHANISM OF ACTION” with Carl P. Weiner, Peter Swaam, and Abhijit Ray as inventors, filed Mar. 24, 2008, which provisional application is incorporated herein by specific reference.

### BACKGROUND OF THE INVENTION

Nitrones constitute a class of compounds that have antioxidant properties due to their ability to form stable adducts (i.e., spin traps) with free radicals. Free radicals can cause oxidative damage to cellular constituents (e.g., proteins and lipids) with pathological consequences. It has been reported that the antioxidant properties of nitrones at least partly underlie their therapeutic potential. Diseases reported to be treatable with antioxidant therapy or that involve free radical generation may also be susceptible to nitrone treatment based on its antioxidant activity. Glutathione (GSH, L-gamma-glutamyl-L-cysteinylglycine) is the predominant endogenous anti-oxidant in the aqueous cytoplasm of biological cells. Virtually all cells require glutathione for function and viability. Glutathione is synthesized in a two-step process from three amino acids, beginning with the combination of glutamic acid and cysteine and ending with the addition of glycine. Glycine and glutamic acids are normally plentiful in cells, so it is the availability of cysteine that is rate limiting. L-cysteine is not very water soluble, nor is it absorbed well by the intestine. Unfortunately, many people may suffer from a lack of cysteine and currently there may not be available ways in which L-cysteine may be effectively delivered to cells.

Glutathione (GSH) is a tripeptide normally found in all animal cells and most plants and bacteria at relatively high concentrations (1-10 millimolar). Glutathione helps protect cells from oxidative damage that would otherwise be caused by free radicals and reactive oxidative intermediates (ROIs) produced during normal cell metabolism, or the result of pathology, for example, drug overdose. Glutathione is the major scavenger of reactive oxidative intermediates in all eukaryotic life forms and is generally required to protect cells against damage by oxidants. Glutathione reduces (and thereby detoxifies) intracellular oxidants. Glutathione is oxidized to the disulfide linked dimer (GSSG) by this reaction, which is actively pumped out of cells and thus largely unavailable for the regeneration of reduced glutathione. As a result, glutathione utilization is associated with a reduction in the amount of glutathione available. For the most part, glutathione must be resynthesized via other metabolic pathways. Glutathione also promotes a net antioxidant balance by maintaining other antioxidants in their reduced forms. Thus, pharmaceutical compounds that replenish or elevate glutathione levels work, at least in part, by enhancing the defense mechanisms utilized normally to protect tissue from ROI mediated damage.

Reactive oxygen species are a natural but undesirable byproduct of cellular metabolic processes within subcellular compartments and membranes. These radicals are highly reactive and destructive to cell tissue because of the presence of unpaired electrons. Free radical reactive oxygen species include but are not limited to O²⁻, OH, H₂O₂, NO, and ONOO. In normal systems, injury from these reactive species is prevented or minimized by radical scavenging systems, including enzymatic systems such as catalase, CuZn-superoxide dismutase, Mn-superoxide dismutase, and glutathione peroxidase. Non-enzymatic radical scavenging systems are also present in metabolic processes, such as glutathione, vitamin E, vitamin C and carotene. With the exception of glutathione, these naturally occurring scavengers are selective in the types of free radicals they can reduce.

The localized deficiency of oxygen severe enough to require anaerobic metabolism is referred to as ischemia. It is a common and important healthcare problem. Transient or intermittent ischemia is characterized by the formation of reactive oxygen species at a greater rate than can be scavenged by the natural, free radical scavenging systems following reperfusion (restoration of blood flow). Ischemia/reperfusion reportedly down regulates antioxidant enzymatic defenses. In response, some suggest that reactive oxygen species are a principal component of the pathologic process that causes cellular injury after an ischemic insult.

Ischemia is associated with multiple clinical conditions. One of them, septic shock, does not involve discrete episodes of reperfusion. It is one of the major causes of death across the lifespan. The manifestations of sepsis include those related to the systemic response to infection (tachycardia, tachypnea alterations in temperature and leukocytosis) and those related to organ-system dysfunction (cardiovascular, respiratory, renal, hepatic and hematologic abnormalities). Lipopolysaccharide (LPS) from the cell wall of gram-negative bacteria is considered the most important exogenous mediator of septic shock. LPS or another endotoxin triggers the release of cytokines and other cellular mediators including tumor necrosis factor a (TNFα), interleukin-1 (IL-1), interleukin-6 (IL-6) and thromboxane A2. Extreme levels of these mediators are known to trigger such pathological events as fever, shock, and intravascular coagulation. The latter obstructs the microcirculation resulting in ischemia and organ failure. Depending on the duration of the ischemia, disturbed cellular metabolism and ion gradients cause irreversible cellular injury and death.

Sickle cell anemia is another cause of ischemia where abnormally shaped red blood cells obstruct the microcirculation. It is the result of a hereditary hemoglobinopathy where the hemoglobin S is present in place of the normal hemoglobin A. Sickle cell crisis is associated with both systemic hypoxia and excess free radical generation.

Organ transplant surgery, where the organ is removed from the donor body, isolated from its blood/oxygen and nutrient supply for an extended period of time and then ‘hooked’ up to the recipient, is also associated with an ischemia reperfusion injury with excess free radical generation that threatens the success of the transplant.

The events that cause reactive oxygen species to be generated after an ischemic episode faster than they can be scavenged are poorly understood. However, the damage caused by these reactive oxygen species is well documented and includes increased intracellular calcium, lipolysis, production of free fatty acids and bioactive arachidonic acid metabolites, proteolysis and decreased intracellular phospholipids. Most of these events are associated with glutathione depletion.

Glutathione depletion is also associated with infection by human immunodeficiency virus (HIV). In HIV infection, the cysteine/glutathione depletion is known to impair T-cell function and is associated with impaired survival of subjects with less than 200 CD4 T-cells per microliter.

Drug toxicity is another widespread problem, and cysteine/glutathione depletion and oxidative stress (See U.S. Pat. No. 4,757,063) intensify drug toxicity and have been implicated in the mechanism of drug toxicity reactions. Acetaminophen, also known as paracetamol and N-acetyl-p-aminophenol, is one of the most widely used pharmaceutical analgesic and antipyretic agents in the world. Acetaminophen is rapidly absorbed from the stomach and small intestine and is normally metabolized by conjugation in the liver to nontoxic agents, which are then eliminated in the urine. It is included in over 100 products and is commonly found in the U.S. as immediate release tablets and as extended-release preparations. Various children's chewable, suspension, and elixir formulations that contain acetaminophen are prevalent. Acetaminophen is also found as a component of combination drugs, such as propoxyphene/acetaminophen and oxycodone acetaminophen. Many companies package acetaminophen under different trade names, resulting in inadvertent overdosing by less sophisticated patients and parents who do not read the information on the packaging. In addition, cold remedies and other over-the-counter preparations often contain acetaminophen, which is listed among a series of generic drug names that are difficult for patients and parents to read. Therefore, patients often are unaware of the amount of acetaminophen that they have received. Children are especially vulnerable to accidental exposure due to their smaller size, the presence of acetaminophen in multiple over-the-counter remedies, and a reluctance to administer aspirin and other NSAIDs to children for fever due to the risk of Reye's Syndrome and renal tubular injury. Acetaminophen is widely used in hospitals for its antipyretic properties. However, acetaminophen may not be the antipyretic agent of choice under circumstances where renal or hepatic function is in danger of being compromised.

Acetaminophen overdose is the single most commonly encountered substance in toxic ingestions, and it is known to be associated with cysteine/glutathione depletion. In many cases, overdose is unintentional and undiagnosed until after substantial damage has occurred. As discussed by Donovan (1999) Academic Emergency Med. 6: 1079-1082, methods for detecting post-ingestion blood levels of acetaminophen suffer poor predictive values. Even in the case of a single acute ingestion, patients still develop toxicity and die absent discernible risk factors for liver injury and low blood levels of acetaminophen. It is well established that acetaminophen overdose causes hepatotoxicity and, in some cases, nephrotoxicity in humans and in experimental animals. Acute overdosage of acetaminophen results in dose-dependent and potentially fatal hepatic necrosis as well as (in rare cases) renal tubular necrosis and hypoglycemia. In acute overdose or when maximum daily dose is exceeded over a prolonged period, the normal pathways of metabolism become saturated.

Excess acetaminophen is metabolized in the liver via the mixed function oxidase P450 system to a toxic, N-acetyl-p-benzoquinone-Imine (NAPQI). NAPQI has an extremely short half-life. Under normal conditions, it is rapidly conjugated with glutathione, a sulfhydryl donor, and removed from the circulation. Under conditions of excessive NAPQI formation or reduced glutathione stores, NAPQI is free to bind to vital proteins and the lipid bilayer of hepatocytes, causing hepatocellular death and subsequently, centrilobular liver necrosis. Immunohistochemical studies have suggested that NAPQI-protein adducts appear even at sub-hepatotoxic acetaminophen doses. In addition, decreased intracellular cysteine/glutathione can contribute to cell death via mechanisms that do not involve NAPQI.

The direct healthcare cost of acetaminophen overdose has been estimated at $87 million annually. Effective protocols have been developed and tested to stratify risk and treat patients who present soon after a single, large dose of acetaminophen. Early treatment of overdosage is considered crucial, and vigorous supportive therapy is essential when intoxication severe. However, many present after a delay long enough for the metabolism of all the acetaminophen, after two or more ingestions over several hours, or after several days of excessive self-medication. Under these circumstances, it is difficult for the clinician to estimate the risk of adverse outcome before hepatic or renal injury occurs. See, for example, Bond and Hite (1999) Acad. Emerg. Med. 6: 1115-1120; and Donovan (1999) Acad. Emerg. Med. 6: 1079-1082.

Nucleoside reverse transcriptase inhibitor such as the pyrimidine nucleoside analogue azidothymidine (AZT, zidovudine are often given to treat HIV as part of combination therapies). Long-term AZT therapy is commonly associated with dose-dependent hematologic toxicity which manifests as low erythrocyte counts and elevated mean red cell volume, and with muscle fiber toxicity, particularly in patients with advanced HIV disease. Some studies teach that AZT's toxic interactions result from the generation of reactive oxygen species which react with and deplete intracellular glutathione levels. (de la Asuncion, et al (1998) J. Clin. Invest. 102 (1): 4-9; Gogu et al. (1991) Exp. Hematol. 19 (7): 649-652; Gogu and Agrawal (1996) Life Sci. 59 (16): 1323-1329; Prakash et al (1997) Arch. Biochem. Biophys. 343 (2): 173-80).

Acetaminophen use in HIV infected patients further lowers glutathione levels and exacerbates AZT toxicity (Richman, et al. (1987) N. Eng. J. Med. 317: 192 97). In vitro and animal studies support this conclusion. AZT treatment also causes oxidative damage to mitochondrial DNA (including increased mitochondrial lipoperoxidation) and increased levels of oxidized glutathione in skeletal muscle in mice (de la Asuncion, et al. (1998) J. Clin. Invest. 102 (1): 4-9). NAC and the anti-oxidant Vitamins C and E each reportedly reduce or prevent this AZT-induced toxicity (Gogu, et al (1991) Exp. Hematol. 19, 649-52; and Gogu and Agrawal (1996) Life Chem. Rep. 4, 1-35). AZT treatment intensifies the rate of glutathione depletion in HIV-TAT transgenic mice (Prakash, 0., et al. Arch. Biochem. Biophys. (1997) 343: 173-80) where expression of the TAT protein depletes glutathione by decreasing glutathione biosynthesis (Choi, J., et al., (2000) J. Biol. Chem. 275 (5): 3693-98) and the activity of antioxidant enzymes (Flores et al. (1993) Proc. Nat. Acad. Sci. 90 (16): 7632-36). Clinical trials of AZT efficacy for the treatment of HIV disease report an association between AZT toxicity and acetaminophen use (Richrnan, et al. (1987) New Eng. J. Med. 317 (4): 192-97. Although the mechanism of this toxic reaction is poorly understood, acetaminophen does not impair AZT detoxification. Thus, in conditions associated with glutathione depletion, such as the later stages of HIV, detoxification of acetaminophen will increase the potential for AZT toxicity, and therapy that counters glutathione depletion may reduce it.

Long-term antibiotic usage may also be associated with drug toxicity reactions. Toxicity is a function of a drugs mechanism of action and its clearance pathways. Scientists have long sought to identify agents that would be generally effective in combating drug toxicity reactions. One recognized treatment for acetaminophen overdose is the administration of sulfhydryl compounds such as L->methionine, L-cysteine, and either the purified L-enantiomer or the racemate mixture of N-acetylcysteine. Also, cimetidine, dimethyl sulfoxide, and ethanol have been shown to inhibit acetaminophen bioactivation. N-acetylcysteine (NAC) is effective in humans when given orally. Early administration of sulfhydryl supplying compounds (0 to 10 hours after acetaminophen ingestion) may prevent or minimize hepatic and renal injury in cases of acetaminophen overdose. NAC is now used by many physicians for treatment of hepatic failure of any etiology, whether known or unknown, and is the accepted antidote for cyclophosphamide poisoning.

It appears that NAC provides the cysteine needed to remediate the excessive sulfur loss associated with HIV disease and specifically to replenish intracellular glutathione. This in turn helps restore the reducing power needed for deoxynucleotide synthesis at a normal rate of cell division. This decreases AZT-mediated inhibition of erythroid development, helps to improve the overall metabolism and stability of erythrocytes and their progenitors. In addition, it improves the cell's ability to withstand the production of oxidants induced by the introduction of drugs (such as AZT) and the internal production of molecules such as TNF and HIV-TAT that trigger intracellular oxidant production.

Researchers have either proposed or utilized various pharmacologic compounds in an attempt to minimize the ischemic insult after reperfusion. Some, such as deferoxamine, allopurinol, catalase, and peroxidase may counteract free radical production. Others, such as superoxide dismutase, destroy these radicals. Still others, such as vitamin E and molecules bearing thiol groups such as N-acetyl cysteine (NAC) and reduced glutathione can neutralize the free radicals. See, e.g., U.S. Pat. No. 5,498,427. WO 88/05044 teaches the use of nitric oxide compounds for the prophylaxis and treatment of ischemic cell damage during perfusion, preservation, and reperfusion of organs in cases of cardioplegia or organ transplantations. The nitric oxides are preferably employed as stable free radicals in their reduced form. U.S. Pat. No. 4,877,810 teaches that the use of the Trolox, a derivative of vitamin E, instead of superoxide dismutase (SOD), reduces cardiac damage upon reperfusion following cardiovascular surgery, including heart transplants. All these therapeutic approaches are less than ideal in preventing ischemia/reperfusion injury for a myriad of reasons. In light of the broad range of pathology and absent effective therapy, there is a need for better means of combating ischemia reperfusion injury. There also exists a need to combat/nullify/eliminate the impact of ischemia caused by diseases and other conditions that are not associated with discrete reperfusion episodes.

The medical crisis following the Chernobyl nuclear accident and the threat of terrorist nuclear attack have raised awareness that high dose, total-body irradiation may occur and cause death due to the gastrointestinal and hematopoietic syndromes. The combination of the prodromal syndrome followed by the gastrointestinal syndrome and bone marrow death induces dehydration, anemia, infection, and ultimately irreversible shock. Current treatment for the subacute gastrointestinal and hematopoietic syndromes includes supportive therapy such as plasma volume expansion, platelets, and antibiotics to prevent dehydration and infection and promote bone marrow repopulation. Human total-body exposure above 10 Gy has been regarded as uniformly fatal. Others suggest that survival may be possible with therapeutic intervention at up to 15 Gy though the symptoms would not be manageable beyond 20 Gy.

A prodromal syndrome followed by three acute or subacute lethal syndromes Mhas been described to be a risk of whole-body radiation exposure after a nuclear accident or a nuclear attack. Death can be expected after very high doses (above 100 Gy) within 24-48 h secondary to neurologic and cardiovascular breakdown. The gastrointestinal syndrome follows 4-10 d after whole-body exposure to 5-12 Gy and is associated with bloody diarrhea and breakdown of the gastrointestinal mucosa. The hematopoietic, or bone marrow syndrome follows whole body exposure between 3-8 Gy and requires several weeks to manifest as the cells of the immune system are lost but not replaced.

The physiological mechanisms of radiation injury have been extensively studied. The systemic damage is partially due to the overproduction of reactive oxygen species (ROS), which disrupt the delicate pro-oxidant/antioxidant balance of tissues leading to protein, lipid, and DNA oxidation. For example, oxidation of the glucosamine synthetase active site sulfhydryl groups is a key factor in the toxicity of the gastrointestinal syndrome. Polyunsaturated fatty acids, when exposed to ROS, can also be oxidized to hydroperoxides that decompose in the presence of metals to hydrocarbons and aldehydes such as MDA. This lipid peroxidation can cause severe impairment of membrane function through increased membrane permeability and membrane protein oxidation. DNA oxidation can lead to strand breakage and consequent mutation or cell death. Glutathione is the principal intracellular thiol responsible for scavenging ROS and maintaining the oxidative balance in tissues.

Thiol supplementation to maintain tissue redox balance has been investigated. Studies of various thiol radioprotectants, such as WR-2721 alone or in combination with prostaglandins, reveal prevention of radiation-induced damage to the intestinal epithelial cells and stem cells. Cysteine and glutathione delivery compounds have been used to protect normal cells from antitumor agents and radiation. The natural L-isomer of NAC (LNAC), a mucolytic agent and the drug of choice in paracetamol/acetaminophen intoxication, replenishes glutathione indirectly through deacetylation to cysteine and directly prevents oxidative damage through scavenging of ROS. LNAC has also been shown to prevent radiation-induced DNA breaks.

Fetal brain damage associated with chronic fetal hypoxemia involves the generation of excess ROI that lead to neuronal loss. This neuronal loss can be blocked by restoration of the oxidative balance using NAC despite the continuation of fetal hypoxemia. Fetal treatment of chronic hypoxemia and other fetal disorders associated with excess free radical production with NAC are compromised by its poor bioavailability.

Thus, drugs that counteract and restore the antioxidant/oxidant balance are of great interest for many disorders. However the limitation of NAC treatment is the requirement of very high concentrations of the compound for treatment.

Therefore it would be desirable to provide an antioxidant that effectively combats oxidation in cells, which may result from various events, such as ischemic events, drug toxicity, radiation, and the like, without requiring a prohibitively high concentration of the compound for treatment. Further, it would be desirable to provide an antioxidant that is capable of acting through a mechanism which more effectively quenches ROIs.

### BRIEF SUMMARY OF THE INVENTION

Generally, the present invention includes antioxidants, compositions containing antioxidants, and method of using the antioxidants in therapies that can respond to treatment with antioxidants.

In one embodiment, the present invention is an antioxidant compound as in Formula 1, and its pharmaceutically acceptable salts, enantiomers, diastearomers, N-oxides, prodrugs or metabolites, or the like:

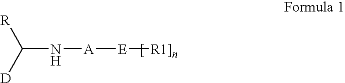

Wherein one or more of R, R1, or D is an antioxidant substituent; R and D are each independently selected from hydrogen or groups containing cysteine, thiols, disulfides, amino acids, amines, amides, or carboxylic acids; when one of R or D is hydrogen, the other includes a thiol, disulfide, or carboxylic acid; A can be CO, SO, SO₂, or C═S; E is one or more ring groups which are aromatic, carbocyclic, and/or heterocyclic 5, 6 and 7 membered rings being mono, bi, tri, tetra, penta, hexa, hepta or octa cyclic fused rings that are substituted or unsubstituted, each heterocyclic ring can include one or more hetero atoms chosen from to O, S, N, Se, or P; each R1 is at a para, meta, and/or ortho position; n is 1, 2, 3, 4, or 5 for each ring; and each R1 is hydrogen, aliphatic, aromatic, and/or an antioxidant substituent.

In one embodiment, the antioxidant substituents are selected from cysteines, nitrones, alkyliminyl oxides, oxaziridines, pyridine oxides, pyridine-1-oxides, piperidinyl-1-oxyls, isoindolines, methyl-oximidazolidine-1-oxyls, 3,5-di-tert-butyl-4-hydroxybenzamido groups, scorbates, derivatives thereof, or combinations thereof.

In one embodiment, E is one or more of Benzene, Naphthalene, Anthracene, Phenanthrene, Benzopyrene, Coronene, Pyrene, Perylene, Triphenylene; Stilbene, Pyridine, Quinoline, Isoquinoline, Pyrazine, Quinoxaline, Acridine, Pyrimidine, Quinazoline, Pyridazine, Cinnoline, Furan, Benzofuran, Isobenzofuran, Pyrrole, Indole, Isoindole, Thiophene, Benzothiophene, Benzo[c]thiophene, Imidazole, Benzoimidazole, Purine, Pyrazole, Indazole, Oxazole, Benzoxazole, Isoxazole, Benzisoxazole, Thiazole, Benzothiazole, 2H-Pyrane, Azulene, isoalloxazine, 1,2,3-Triazine, 1,2,4-Triazine, 1,3,5-Triazine, 2,4,6-Triphenylphosphorine, or thiophene.

In one embodiment, each R1 is independently selected from the group H, F, Cl, Br, I, —OH, —CF₃, —OR2, —CN, —NO2, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, —C(NO)C(CH₃)₃, oxaziridine ring, nitrone or ozaziridine ring with C1-C6 straight or branched substituted or unsubstituted saturated or unsaturated alkyl, where p can be 0, 1, or 2, and R2 is an aromatic or an aliphatic group.

In one embodiment, when included, R2, at each occurrence, is independently selected from the group H, OH, C1-C6 alkyl, C2-C6 alkenyl, C2-C6 alkynyl, 5-7 membered saturated or unsaturated aromatic or carbocycle that is substituted or unsubstituted, 5-7 membered saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C3-C14 saturated or unsaturated aromatic or carbocycle that is substituted or unsubstituted, —C(O)C3-14 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)O-5-7 membered saturated or unsaturated aromatic or carbocycle that is substituted or unsubstituted and with one or more heteroatoms. For example, the heteroatoms are nitrogen, oxygen, sulfur, or selenium.

In one embodiment, when substituted, R2 is substituted with R3, which at each occurrence, is independently selected from the group F, Cl, Br, OH, I, ═O, ═S, ═NR4, ═NOR4, ═N—NR4R4, —CF₃, —OR4, —CN, —NO₂, —NR4R4, —C(O)R4, —C(O)OR4, —OC(O)R4, —C(O)NR4R4, —NR4C(O)R4, —OC(O)NR4R4, —NR4C(O)OR4, —NR4C(O)NR4R4, —C(S)R4, —C(S)OR4, —OC(S)R4, —C(S)NR4R4, —NR4C(S)R4, —OC(S)NR4R4, —NR4C(S)OR4, —NR4C(S)NR4R4, —C(NR4)R4, —C(NR4)OR4, —OC(NR4)R4, —C(NR4)NR4R4, —NR4C(NR4)R4, —OC(NR4)NR4R4, —NR4C(NR4)OR4, —NR4C(NR4)NR4R4, S(O)ₚR4, —SO₂NR4R4, —R4, —C(NO)C(CH₃)₃, oxaziridine ring, nitrone or ozaziridine ring with C1-C6 straight or branched substituted or unsubstituted saturated or unsaturated alkyl, wherein R4 is an aromatic or aliphatic group, substituted or unsubstituted.

In one embodiment, when included R4, at each occurrence, is independently selected from the group H, C1-C6 alkyl, C2-C6 alkenyl, C2-C6 alkynyl, 5-7 membered saturated or unsaturated aromatic or carbocycle that is substituted or unsubstituted, 5-7 membered member saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C5-C7 saturated or unsaturated aromatic or carbocycle that is substituted or unsubstituted, —C(O)C5-C7 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)OC5-C7 saturated or unsaturated aromatic or carbocycle that is substituted or unsubstituted and with one or more heteroatoms. The heteroatoms can be nitrogen, oxygen, sulfur, and selenium, or the like.

In one embodiment, each of R or D is independently hydrogen, F, Cl, Br, I, —OH, —CF₃, —CH₂SH, —(CH₂)ₘSH, —(C1-C6 alkyl)-thiol, —(C1-C6 alkyl)disulfide, —CH₂-disulfide, —(CH₂)ₘ-disulfide, C(O)OH, —(C1-C6 alkyl)-C(O)OH, —OC1-C6 alkyl, —SH, —SC1-C6 alkyl, —CN, —NO2, —NH2, —NHC1-C6 alkyl, —N(C1-C6 alkyl)₂, —C(O)C1-C6 alkyl, —OC(O)C1-C6 alkyl, —C(O)OC1-C6 alkyl, —C(O)NH₂, —C(O)NHC1-C6 alkyl, —C(O)N(C1-C6 alkyl)₂, —NHC(O)C1-C6 alkyl, —SO₂NH₂, —SO₂NHC1-C6 alkyl, —SO₂N(C1-C6 alkyl)₂, and —S(O)ₚC1-C6 alkyl, 4-carboxyphenyl, amino acid, amine, or amide, each alkyl being substituted or unsubstituted, wherein m is 1, 2, 3, 4, 5, 6, 7, 8, 9, or 10.

In one embodiment, R1 can include a sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy, or substituted carboxy.

In one embodiment, R1 is CH₂CH₂COL, where L is OCH2CH(OH)M, and M is

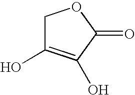

### or E.

In one embodiment, R1 is

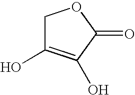

or derivative thereof.

In one embodiment, R1 is

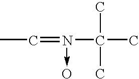

or derivative thereof.

In one embodiment, when an R1 is not an antioxidant it is independently selected from the group H, OH, F, Cl, Br, I, —CF₃, —OR2, —CN, —NO₂, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, or the like. R2 can be an aliphatic or aromatic.

In one embodiment, the antioxidant is a cysteine nitrone. For example, the antioxidant has a chemical structure of Formula 2:

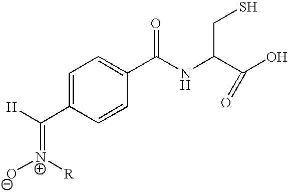

wherein the R group of Formula 2 includes at least one of an alkyl, ring, alkyliminyl oxide, aliphatic, aromatic, and/or aryl group.

In one embodiment, the alkyl or aliphatic group of the antioxidant can be straight, branched, substituted, unsubstituted, saturated, unsaturated, and where the ring, aromatic, or aryl group can be fused, linked, homoatom, heteroatom, substituted, unsubstituted, saturated, unsaturated, or the like.

In one embodiment, the antioxidant is a cysteine nitrone compound having Formula 3, Formula 4, Formula 5, Formula 6, Formula 7, as shown and defined below:

In one embodiment, the antioxidant is present in a therapeutically effective amount in a subject. The subject can have a condition that can be treated, inhibited, and/or prevented with antioxidant therapy.

In one embodiment, the subject has the antioxidant in a therapeutically effective amount for: treating, inhibiting, and/or preventing bronchopulmonary disease, asthma, diabetes mellitus, myocardial infarction, damage caused by drug abuse and/or overdose, acetaminophen toxicity, alcoholism, burn injury, acute radiation exposure, Alzheimer's disease, Parkinson's disease, cerebral ischemia, cerebral stroke, traumatic brain injury, acute spinal cord injury, alopecia, aging, inflammatory bowel disease, autoimmune disorders, preterm parturition, premature cervical ripening, pregnancy loss, cerebrovascular stroke, retinal ischemia, macular degeneration, degenerative disorders of the retina, renal ischemia, arteriosclerosis, cardiovascular diseases, amyotrophic latral sclerosis, Huntington's disease, multiple sclerosis, head trauma, nerve injury, neuropathies, migraine, schizophrenia, mood disorders, pancreatitis, pancreatic disorders, diabetes, epilepsy, transplant and graft failure or rejection, hepatitis, jaundice-induced liver disorders, lung injury or damage, gastric ulcer, endotoxemia, aging or senescence, preterm labor, fetal damage due to intrauterine ischemia, pain syndromes acute and chronic or neuropathic, arthritis, autoimmune disorders, asthma, allergic reactions, inflammatory bowel disease, irritable bowel syndrome, uveitis, cancer, complications and disorders arising from cancer therapy, alopecia, fetal inflammatory syndrome, arthritis, uveitis, obesity, eating disorders, cancer, sleeping disorders, cognition, depression, anxiety, high blood pressure, lipid disorders and atherosclerosis, abnormal intrauterine conditions; newborn brain abnormalities; intrauterine oxygen deficiency, either chronic or acute; perinatal brain damage; acute oxygen depravation; perinatal hypoxic ischemic reperfusion injury; brain damage associated with acute oxygen depravation; conditions associated with primary cell death due to lack of oxygen; conditions associated with secondary cell death due to inflammatory response by reperfusion with oxygenated blood; acute ischemic reperfusion during labor; cerebral palsy; intrauterine growth restriction (IUGR); chronic fetal hypoxemia; fetal inflammatory response in the brain; preterm labor and/or birth associated with fetal inflammatory response; idiopathic preterm birth; or other condition caused by excessive oxidation, or combinations thereof; or modifying mammalian inflammatory pathways.

In one embodiment, the antioxidant is present in a pharmaceutical composition. The antioxidant can be present in the pharmaceutical composition in a therapeutically effective amount. The pharmaceutical composition can further include a pharmaceutically acceptable carrier.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an embodiment of a reaction scheme for synthesizing an antioxidant of the present invention.

FIG. 2 illustrates an embodiment of a reaction scheme for synthesizing an antioxidant of the present invention.

FIG. 3 illustrates various antioxidant embodiments of the invention, where A is as shown in the top of the figure (1-carboxy-2-mercaptoetylcarbamoyl).

FIG. 4 illustrates an embodiment of a reaction scheme for synthesizing an antioxidant of the present invention.

FIG. 5 illustrates an embodiment of a reaction scheme for synthesizing an antioxidant of the present invention.

FIG. 6 illustrates an embodiment of a reaction scheme for synthesizing an antioxidant of the present invention.

FIG. 7 illustrates an embodiment of a reaction scheme for synthesizing an antioxidant of the present invention.

FIG. 8 illustrates an embodiment of a reaction scheme for synthesizing an antioxidant of the present invention.

FIG. 9 illustrates an embodiment of a reaction scheme for synthesizing an antioxidant of the present invention.

### DETAILED DESCRIPTION OF THE PREFERRED EMBODIMENTS

An antioxidant is a molecule capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols (glutathione), tocopherols, tocotrienols (vitamin E) or polyphenols (resveratrol). There is ample evidence in the literature that nitrone-based compounds can act as free radical traps thereby conferring antioxidant properties. Since antioxidant activity is a purely chemical reaction, it is clear that the compounds in the present disclosure containing two known antioxidant chemical moieties (thiols and nitrones) tethered by an inert linker group must have strong antioxidant properties in of themselves.

The amount of protection provided by any one antioxidant will also depend on its concentration, its reactivity towards the particular reactive oxygen species being considered, and the status of the antioxidants with which it interacts. Therefore, the antioxidant activity is strongly dependent on the ability to enter the cell, i.e. its level of cellular penetration. In general, compounds with relatively high cellular permeability and concomitant high oral bioavailability, confer antioxidant protection at lower systemic concentrations and can be administered at lower doses than compounds with relatively poor cell membrane penetration. There is a strong correlation between membrane permeability, oral bioavailability and the solubility and lipophilicity of a compound (expressed as log P), which can be calculated accurately from molecular structure. In general, compounds with a log P value between −2 and +3 have good membrane permeability. The compounds in the present disclosure have logP values ranging between −1.0 to approximately 1.5. Furthermore, we have recently shown that computational models can be used efficiently to categorize drug molecules into 4 separate classes of the Biopharmaceutics and Drug Disposition Classification System (BDDCS), a system used by the FDA to confer biowaivers for newly marketed generic preparations. According to our models, the chemical entities proposed in the present application would fall into BDDCS Class I. Compared to other drugs already on the US market, Class I compounds would possess high bioavailability (i.e. >85% of the total dose absorbed), making them excellent candidates for oral administration of antioxidants.

The present invention relates to the field of novel molecules that function as antioxidants and specifically to their use as pharmaceutical agents to treat oxidative stress caused by but not limited to: bronchopulmonary disease, asthma, diabetes mellitus, myocardial infarction, damage caused by drug abuse and or overdose, alcoholism, burn injury whether accidental or the result of a chemical accident/attack, acute radiation exposure whether from nuclear attack or nuclear accident, anti-HIV therapy, Alzheimer's disease, Parkinson's disease, providing protection against cerebral ischemia whether after cerebral stroke or before or after birth in the fetus/perinate, preventing damage caused by mutation of DNA resulting in cancer, uveitis, the treatment and prophylaxis of pain syndromes (acute and chronic or neuropathic), traumatic brain injury, acute spinal cord injury, alopecia (hair loss), aging, inflammatory bowel disease and autoimmune disorders modulating inflammatory response, and as a therapeutic adjuvant for the treatment—of preterm parturition, premature cervical ripening leading to pregnancy loss. Also, the novel antioxidants can be used in therapies for any ailment or condition described in the background section of this document.

In one embodiment, the antioxidant can be cysteine nitrones or derivatives thereof containing a nitrone and/or cysteine and/or other substituents that can function as an antioxidant, such as a spin trap substituent. As such, the molecules of the present invention have antioxidant properties.

In one embodiment, the novel antioxidants are cysteine nitrone compounds or derivatives thereof that include antioxidant substituents. The antioxidants can be used in methods of therapy that may provide improved antioxidant activity compared to already known and available compounds, such as alpha-phenyl-N-tert butyl nitrone (PBN) and N-acetyl cysteine (NAC). Accordingly, the antioxidant compounds of the present invention can be substitutes for PBN and/or NAC, and can be used in any therapy that these other agents are used in. The antioxidant compounds can also be administered with PBN and/or NAC. However, the antioxidents of the present invention, such as those shown by Formula 1 below, are not alpha-phenyl-N-tert butyl nitrone (PBN) and N-acetyl cysteine (NAC). U.S. Pat. No. 6,852,698 is incorporated herein by specific reference.

The antioxidant compounds of the present invention may lower toxicity and enhance water solubility, making these compounds more readily available to the affected tissue. The novel antioxidant compounds of the present invention may allow for supplementation with cysteine nitrones for boosting intracellular glutathione by increasing intracellular cysteine, while the addition of a nitrone molecule increases the half life of the free radical (i.e., stabilizes it), thereby assisting in ameliorating its adverse effects. Thus, the novel antioxidant compounds of the present invention may utilize a dual mechanism of action which may allow the compounds to more effectively quench ROIs.

### I. Compounds

The present invention includes antioxidants. Generally, an antioxidant compound of the present invention may be Formula 1, and its pharmaceutically acceptable salts, enantiomers, diastearomers, N-oxides, prodrugs or metabolites, or the like.

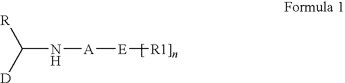

In Formula 1, one or more of R, R1, or D is an antioxidant substituent having radical neutralization capabilities.

In Formula 1, R and D can be independently selected from hydrogen or groups containing cysteine, thiols, disulfides, amino acids, amines, amides, or carboxylic acids.

In Formula 1, when one of R or D is hydrogen, the other includes a thiol, disulfide, or carboxylic acid.

In Formula 1, A can be CO, SO, SO₂, C═S, or the like.

In Formula 1, E can be one or more ring groups which are aromatic, carbocyclic, and/or heterocyclic 5, 6 and 7 membered rings being mono, bi, tri, tetra, penta, hexa, hepta or octa cyclic fused rings that are substituted or unsubstituted. Each heterocyclic ring can include one or more hetero atoms chosen from to O, S, N, Se, P, or the like.

In Formula 1, each R1 can be at a para, meta, and/or ortho position.

In Formula 1, n is 1, 2, 3, 4, or 5 for each ring.

In Formula 1, each R1 can be hydrogen, an aliphatic, aromatic, or an antioxidant substituent.

In Formula 1, the antioxidant substituents can include cysteines, nitrones, alkyliminyl oxides, oxaziridines, pyridine oxides, pyridine-1-oxides, piperidinyl-1-oxyls, isoindolines, methyl-oximidazolidine-1-oxyls, 3,5-di-tert-butyl-4-hydroxybenzamido groups, scorbates, radical-scavenging sulfur groups, radical-scavenging phosphorous groups, or the like.

In Formula 1, examples of E can include Benzene, Naphthalene, Anthracene, Phenanthrene, Benzopyrene, Coronene, Pyrene, Perylene, Triphenylene; Stilbene, Pyridine, Quinoline, Isoquinoline, Pyrazine, Quinoxaline, Acridine, Pyrimidine, Quinazoline, Pyridazine, Cinnoline, Furan, Benzofuran, Isobenzofuran, Pyrrole, Indole, Isoindole,Thiophene, Benzothiophene, Benzo[c]thiophene, Imidazole, Benzoimidazole, Purine, Pyrazole, Indazole, Oxazole, Benzoxazole, Isoxazole, Benzisoxazole, Thiazole, Benzothiazole, 2H-Pyrane, Azulene, isoalloxazine, 1,2,3-Triazine, 1,2,4-Triazine, 1,3,5-Triazine, 2,4,6-Triphenylphosphorine, thiophene or the like.

In Formula 1, when R1 may be independently selected from the group consisting of H, F, Cl, Br, I, —OH, —CF₃, —OR2, —CN, —NO₂, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, —C(NO)C(CH₃)₃, oxaziridine ring, nitrone or oxaziridine ring with C1-C6 straight or branched substituted or unsubstituted saturated or unsaturated alkyl, or the like. In Formula 1, p can be 0, 1, or 2. In Formula 1, R2 can be an aromatic or an aliphatic group. As shown herein, parentheses “(_)” denote a double bond from a carbon to the atom or group displayed within the parentheses. For example, C(O) is C═O, C(S) is C═S, and C(NR2) includes a carbon doubled bonded to the nitrogen which is bonded to R2. The substituents on the nitrone or oxazaridine ring can be traditional substituents such as any substituent described herein.

In Formula 1, when R1 includes R2, R2, at each occurrence, may be independently selected from the group consisting of: H, OH, C1-C6 alkyl, C2-C6 alkenyl, C2-C6 alkynyl, 5-7 membered saturated or unsaturated aromatic or carbocycle that is substituted or unsubstituted, 5-7 membered saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C3-C14 saturated or unsaturated aromatic or carbocycle that is substituted or unsubstituted, —C(O)C3-14 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)O-5-7 membered saturated or unsaturated aromatic or carbocycle that is substituted or unsubstituted and with one or more heteroatoms. The heteroatoms can be nitrogen, oxygen, sulfur, and selenium, or the like. When substituted, R2 can be substituted with R3.

In Formula 1, R or D can independently be hydrogen, F, Cl, Br, I, —OH, —CF₃, —CH₂SH, —(CH₂)ₘSH, —(C1-C6 alkyl)-thiol, —(C1-C6 alkyl)disulfide, —CH₂-disulfide, —(CH₂)ₘ-disulfide, C(O)OH, —(C1-C6 alkyl)-C(O)OH, —OC1-C6 alkyl, —SH, —SC1-C6 alkyl, —CN, —NO2, —NH2, —NHC1-C6 alkyl, —N(C1-C6 alkyl)₂, —C(O)C1-C6-alkyl, —OC(O)C1-C6 alkyl, —C(O)OC1-C6 alkyl, —C(O)NH₂, —C(O)NHC1-C6 alkyl, —C(O)N(C1-C6 alkyl)₂, —NHC(O)C1-C6 alkyl, —SO₂NH₂, —SO₂NHC1-C6 alkyl, —SO₂N(C1-C6 alkyl)₂, and —S(O)ₚC1-C6 alkyl, 4-carboxyphenyl, amino acid, amine, amide, or the like. As shown herein, “C1-C6 alkyl” indicates an alkyl, alkene, or alkyne having from 1 to 6 carbons, which can be saturated or unsaturated and branched or straight chain that is either substituted or unsubstituted. In Formula 1, m can be 1, 2, 3, 4, 5, 6, 7, 8, 9, or 10. The substituents can be traditional substituents such as any substituent described herein. R or D can independently substituted with R2.

In one embodiment, the R2 substituent can also include one or more R3 groups as an additional substituent. In Formula 1, R3, at each occurrence, may be independently selected from the group consisting of: F, Cl, Br, OH, I, ═O, ═S, ═NR4, —NOR4, ═N—NR4R4, —CF₃, —OR4, —CN, —NO₂, —NR4R4, —C(O)R4, —C(O)OR4, —OC(O)R4, —C(O)NR4R4, —NR4C(O)R4, —OC(O)NR4R4, —NR4C(O)OR4, —NR4C(O)NR4R4, —C(S)R4, —C(S)OR4, —OC(S)R4, —C(S)NR4R4, —NR4C(S)R4, —OC(S)NR4R4, —NR4C(S)OR4, —NR4C(S)NR4R4, —C(NR4)R4, —C(NR4)OR4, —OC(NR4)R4, —C(NR4)NR4R4, —NR4C(NR4)R4, —OC(NR4)NR4R4, —NR4C(NR4)OR4, —NR4C(NR4)NR4R4, S(O)ₚR4, —SO₂NR4R4, —R4, —C(NO)C(CH₃)₃, oxaziridine ring, nitrone or ozaziridine ring with C1-C6 straight or branched substituted or unsubstituted saturated or unsaturated alkyl, or the like. In Formula 1, R4 can be an aromatic or an aliphatic group, substituted or unsubstituted. The substituents on the nitrone or oxaziridine ring can be traditional substituents such as any substituent described herein.

In one embodiment, the R3 substituent can include one or more R4 groups as an additional substituent. In Formula 1, R4, at each occurrence, may be independently selected from the group consisting of: H, C1-C6 alkyl, C2-C6 alkenyl, C2-C6 alkynyl, 5-7 membered saturated or unsaturated aromatic or carbocycle that is substituted or unsubstituted, 5-7 membered member saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C5-C7 saturated or unsaturated aromatic or carbocycle that is substituted or unsubstituted, —C(O)C5-C7 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)OC5-C7 saturated or unsaturated aromatic or carbocycle that is substituted or unsubstituted and with one or more heteroatoms. The heteroatoms can be nitrogen, oxygen, sulfur, and selenium, or the like.

In one embodiment, R1 can be a sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy, or substituted carboxy

In one embodiment, any of R1, R2, R3, and/or R4 can include additional substituents. The additional substituents can be any of the substituents described herein.

Additionally, Formula 1 can include R1 being CH₂CH₂COL, where and L is OCH2CH(OH)M. In turn, R1 and/or M can be

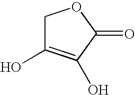

### or E. Also, R1 can be

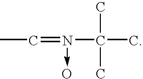

Aliphatics are not aromatic, but can be cyclic, and can alkyls and include any of at least H, C1-C6 alkyl, C2-C6 alkenyl, C2-C6 alkynyl, 3-14 member saturated or unsaturated carbocycle that is substituted or unsubstituted, 3-14 member saturated or unsaturated carbocycle that is substituted or unsubstituted and with one or more heteroatoms, —C(O)C1-C6 alkyl, —C(O)C2-C6 alkenyl, —C(O)C2-C6 alkynyl, —C(O)C3-C14 saturated or unsaturated carbocycle that is substituted or unsubstituted, —C(O)C3-14 saturated or unsaturated carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC3-14 saturated or unsaturated carbocycle that is substituted or unsubstituted, —C(O)OC3-14 saturated or unsaturated carbocycle that is substituted or unsubstituted and with one or more heteroatoms. The heteroatoms can be nitrogen, oxygen, sulfur, phosphorous, and selenium, or the like.

In Formula 1, when R1 is not an antioxidant it may be independently selected from the group consisting of H, F, Cl, Br, I, —CF₃, —OR2, —CN, —NO₂, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, or the like. R2 can be an aliphatic or aromatic.

In one embodiment, the antioxidant is a cysteine nitrone with a chemical structure of Formula 2 or derivative thereof, as well as any acceptable salts, enantiomers, diastearomers, N-oxides, prodrugs or metabolites, or the like. As shown, the cysteine can be substituted onto the molecule opposite of the nitrone. This presents two antioxidant features being presented for radical scavenging.

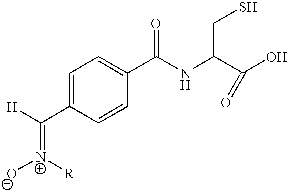

In Formula 2, the R group on this can be any type of one or more alkyl, ring, alkyliminyl oxide, aliphatic, aromatic, and/or aryl groups. The alkyls or aliphatics can be straight, branched, substituted, unsubstituted, saturated, unsaturated, or the like, where the ring, aromatic or aryl groups can be fused, linked, homoatom, heteroatom, substituted, unsubstituted, saturated, unsaturated, or the like. The R group can have chemical groups in series, alternating, or branched with one or more alkyl, ring, and/or aryl groups. When R=Me is (E)-N-(4-(1-carboxy-2-mercaptoethylcarbamoyl)benzylidene)methanamine oxide.

In an embodiment, the cysteine nitrone compound may include one or more of the following Formula 3, Formula 4, Formula 5, Formula 6, Formula 7, or combinations thereof.

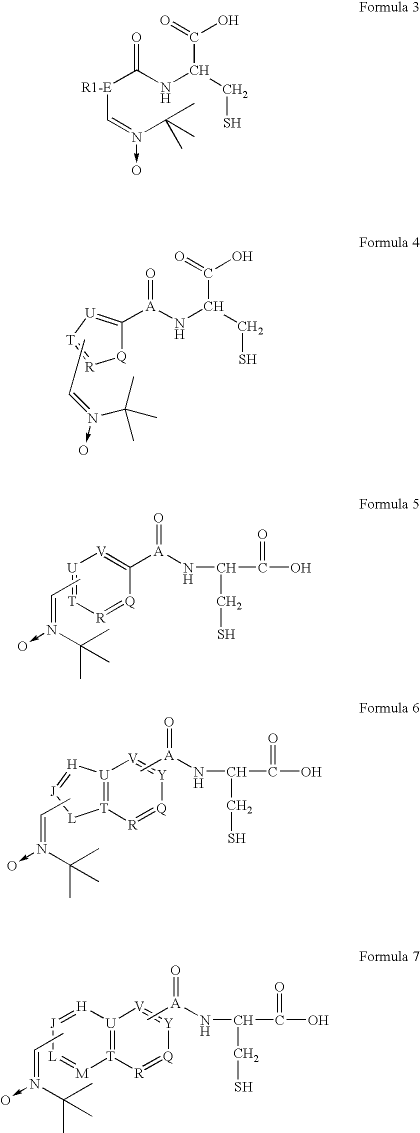

In Formula 3, Formula 4, Formula 5, Formula 6, Formula 7, the substituents H, J, L, M, Q, R, T, U, V may represent CH₂, O, NH, S, CH, and N, and A may represent C or S. H is a substituent when present at a member of a ring group.

Accordingly, the cysteine nitrone compounds of the present invention may include a cycloalkyl, carbocycle, aromatic, cycloheteroalkyl or heteroaryl ring of 5 to 8 atoms, or include bicyclic aryl nitrone compounds that comprise a bicycloalkenyl, bicycloheteroalkenyl, bicycloaryl or bicycloheteroaryl ring of 8 to 11.

In one embodiment, a first position of the ring may be bonded to the carbon atom of a nitrone group. The carbon atom of the nitrone may be further bonded to hydrogen, substituted or unsubstituted (C1-C6) alkyl, substituted or unsubstituted (C1-C6) cycloalkyl, substituted or unsubstituted aryl, or substituted or unsubstituted arylalkyl. The nitrogen atom of the nitrone group may be bonded to a substituted or unsubstituted aliphatic, substituted or unsubstituted alkyl, substituted or unsubstituted heteroalkyl, substituted or unsubstituted acetyl, substituted or unsubstituted aryl, substituted or unsubstituted heteroaryl, substituted or unsubstituted arylalkyl, or substituted or unsubstituted heteroarylalkyl. A second position of the ring, which may be any position in the cycloheteroalkenyl or heteroaryl ring discussed above, can be linked to a second group, which may be selected form carbonyl, sulfoxyl and sulfonyl groups which is further attached to the N-terminal of a cysteine group.

The third and subsequent substitution in the heteroaryl ring may be selected from hydrogen, sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy and substituted carboxy (i.e., ester). In some embodiments the ring is further substituted, while in other embodiments the ring may be only substituted at two positions.

In particular embodiments, the second and third or first and third groups may be similar. It is contemplated that the third group may bond with the ring at various other positions without departing from the scope and spirit of the present invention. Further, it is contemplated that the ring may be substituted with more than three groups.

Examples of antioxidants of the present invention can include: 2-[4-(tert-Butylnitrone)-benzoylamino]-3-mercapto-propionic acid

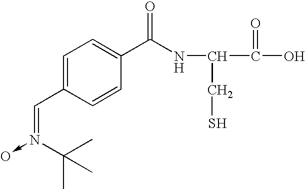

(R,Z)-N-(4-(1-carboxy-2-mercaptoethylcarbamoyl)benzylidene)-2-methylpropan-2-amine oxide

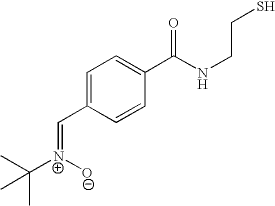

(Z)-N-(4-(carboxymethylcarbamoyl)benzylidene)-2-methylpropan-2-amine oxide

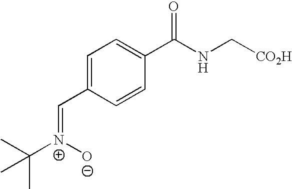

(R)-2-benzamido-3-mercaptopropanoic acid

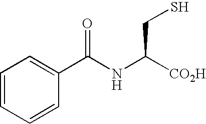

(R,Z)-N-(4-(1-carboxy-2-mercaptoethylcarbamoyl)benzylidene)methanamine oxide

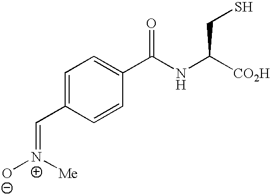

(2R)-2-(4-(2-tert-butyl-1,2-oxaziridin-3-yl)benzamido)-3-mercaptopropanoic acid

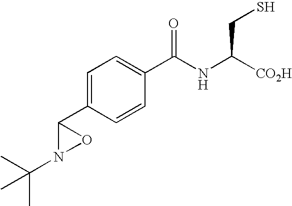

(R,Z)-N-(4-(1-carboxy-2-mercaptoethylcarbamoyl)benzylidene)-2-methylpropan-2-amine oxide

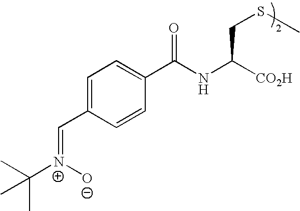

4-(4-(1-carboxy-2-mercaptoethylcarbamoyl)phenyl)pyridine-1-oxide

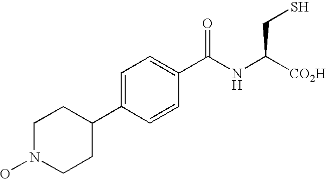

2-(3,5-di-tert-butyl-4-hydroxybenzamido)-3-mercaptopropanoic acid

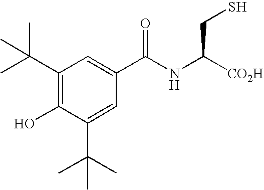

(R,Z)-N-4-(1-(4-carboxyphyenyl)-2-mercaptoethylcarbamoyl)benzylidene)-2-methylpropan-2-amine oxide.

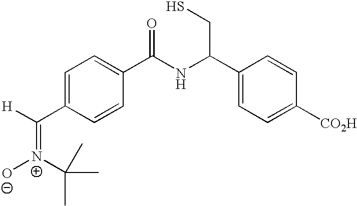

2-[2-(tert-Butylnitrone)-benzoylamino]-3-mercapto-propionic acid

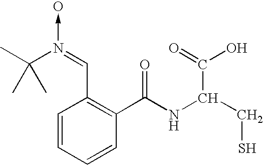

2-{[6-(tert-Butylnitrone)-pyridine-3-carbonyl]-amino}-3-mercapto-propionic acid

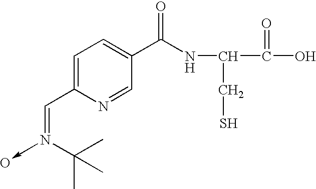

2-{[5-(tert-Butylnitrone)-pyridine-2-carbonyl]-amino}-3-mercapto-propionic acid

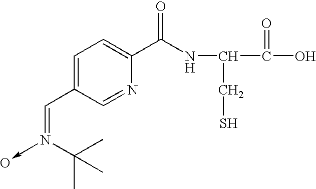

2-{[2-(tert-Butylnitrone)-pyrimidine-5-carbonyl]-amino}-3-mercapto-propionic acid

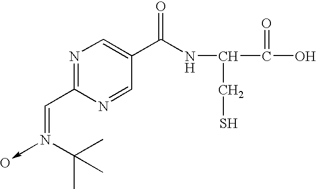

2-{[6-(tert-Butylnitrone)-pyridazine-3-carbonyl]-amino}-3-mercapto-propionic acid

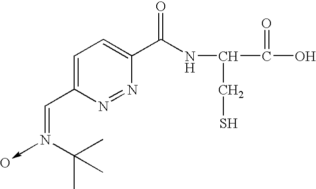

2-{[5-(tert-Butylnitrone)-thiophene-2-carbonyl]-amino}-3-mercapto-propionic acid

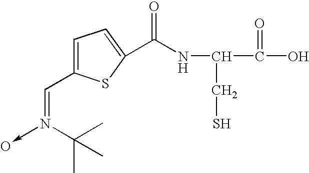

2-{[4-(tert-Butylnitrone)-thiophene-2-carbonyl]-amino}-3-mercapto-propionic acid

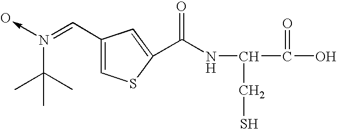

2-{[5-(tert-Butylnitrone)-furan-2-carbonyl]-amino}-3-mercapto-propionic acid

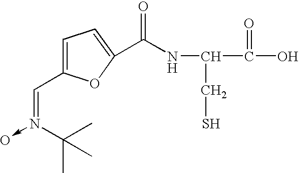

2-{[5-(tert-Butylnitrone)-1-H-pyrrole-2-carbonyl]-amino}-3-mercapto-propionic acid

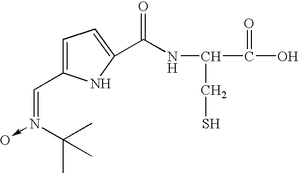

2-{[5-(tert-Butylnitrone)-1-methyl-pyrrole-2-carbonyl]-amino}-3-mercapto-propionic acid

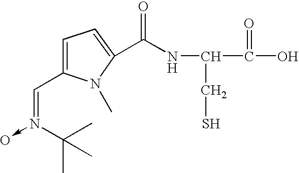

2-{[8-(tert-Butylnitrone)-2,4-dioxo-2,3,4,10-tetrahydro-benzo[g]pteridine-6-carbonyl]-amino}-3-mercapto-propionic acid

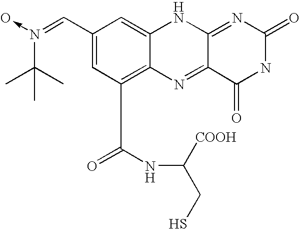

2-{[7-(tert-Butylnitrone)-naphthalene-2-carbonyl]-amino}-3-mercapto-propionic acid

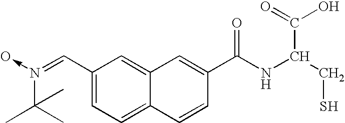

2-{[7-(tert-Butylnitrone)-anthracene-2-carbonyl]-amino}-3-mercapto-propionic acid

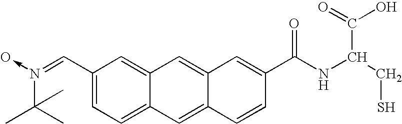

2-{[5-(tert-Butylnitrone)-pyridine-3-carbonyl]-amino}-3-mercapto-propionic acid

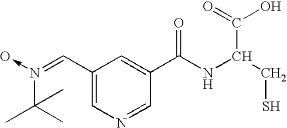

2-{[3-(tert-Butylnitrone)-quinoline-6-carbonyl]-amino}-3-mercapto-propionic acid

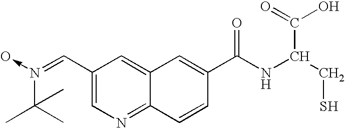

2-{[6-(tert-Butylnitrone)-quinoline-3-carbonyl]-amino}-3-mercapto-propionic acid

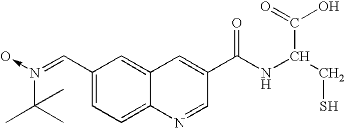

2-{[3-(tert-Butylnitrone)-isoquinoline-6-carbonyl]-amino}-3-mercapto-propionic acid

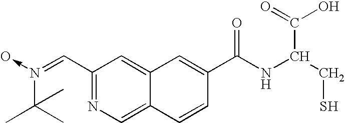

2-{[6-(tert-Butylnitrone)-isoquinoline-3-carbonyl]-amino}-3-mercapto-propionic acid

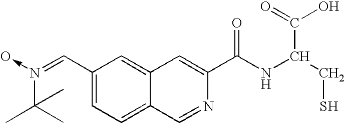

2-{[6-(tert-Butylnitrone)-pyrazine-2-carbonyl]-amino}-3-mercapto-propionic acid

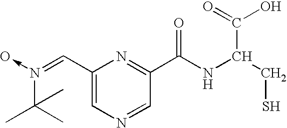

2-{[7-(tert-Butylnitrone)-quinoxaline-2-carbonyl]-amino}-3-mercapto-propionic acid

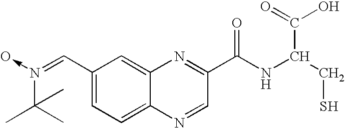

2-{[7-(tert-Butylnitrone)-acridine-2-carbonyl]-amino}-3-mercapto-propionic acid

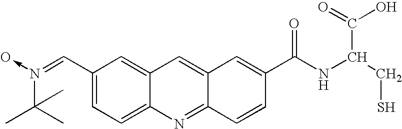

2-{[5-(tert-Butylnitrone)-pyridazine-3-carbonyl]-amino}-3-mercapto-propionic acid

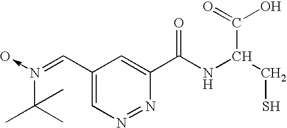

2-{[4-(tert-Butylnitrone)-pyrimidine-2-carbonyl]-amino}-3-mercapto-propionic acid

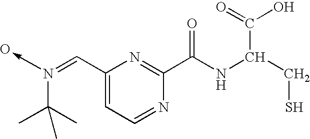

2-{[7-(tert-Butylnitrone)-quinazoline-2-carbonyl]-amino}-3-mercapto-propionic acid

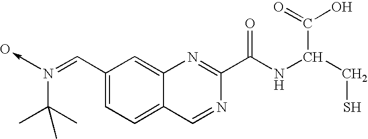

2-{[6-(tert-Butylnitrone)-cinnoline-3-carbonyl]-amino}-3-mercapto-propionic acid

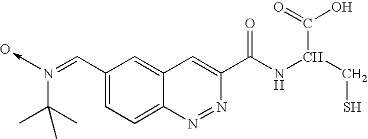

2-{[2-(tert-Butylnitrone)-7aH-indene-5-carbonyl]-amino}-3-mercapto-propionic acid

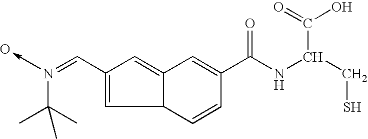

2-{[2-(tert-Butylnitrone)-2H-indole-5-carbonyl]-amino}-3-mercapto-propionic acid

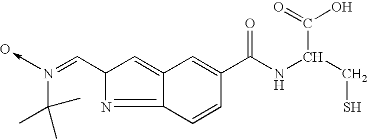

2-{[3-(tert-Butylnitrone)-3aH-isoindole-5-carbonyl]-amino}-3-mercapto-propionic acid

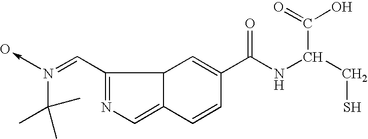

2-{[2-(tert-Butylnitrone)-1H-indole-5-carbonyl]-amino}-3-mercapto-propionic acid

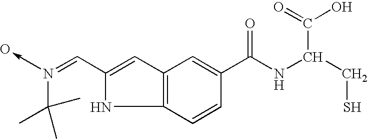

2-{[2-(tert-Butylnitrone)-benzo[b]thiophene-5-carbonyl]-amino}-3-mercapto-propionic acid

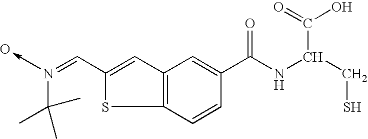

2-{[1-(tert-Butylnitrone)-benzo[c]thiophene-5-carbonyl]-amino}-3-mercapto-propionic acid

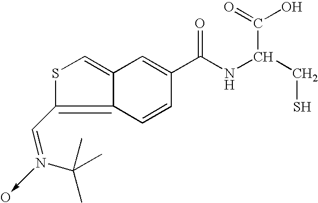

2-{[2-(tert-Butylnitrone)-2H-benzoimidazole-5-carbonyl]-amino}-3-mercapto-propionic acid

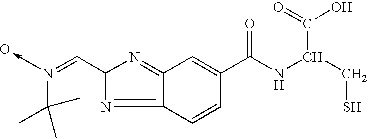

2-{[8-(tert-Butylnitrone)-8H-purine-2-carbonyl]-amino}-3-mercapto-propionic acid

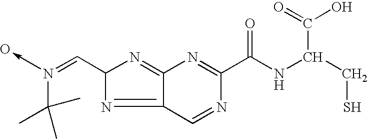

2-{[2-(tert-Butylnitrone)-benzooxazole-5-carbonyl]-amino}-3-mercapto-propionic acid

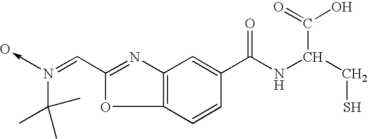

2-{[3-(tert-Butylnitrone)-2H-indazole-6-carbonyl]-amino}-3-mercapto-propionic acid

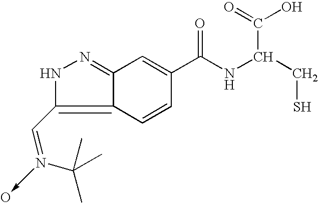

2-{[3-(tert-Butylnitrone)-benzo[d]isoxazole-6-carbonyl]-amino}-3-mercapto-propionic acid

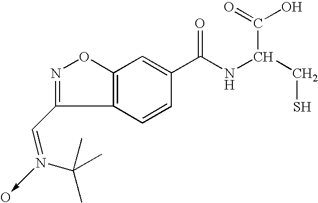

2-{[3-(tert-Butylnitrone)-benzo[d]isothiazole-6-carbonyl]-amino}-3-mercapto-propionic acid

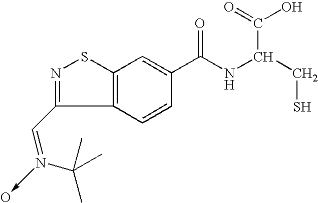

2-{[5-(tert-Butylnitrone)-[1,2,4]triazine-3-carbonyl]-amino}-3-mercapto-propionic acid

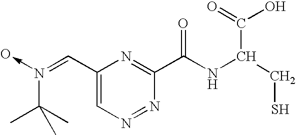

2-{[6-(tert-Butylnitrone)-[1,2,3]triazine-4-carbonyl]-amino}-3-mercapto-propionic acid

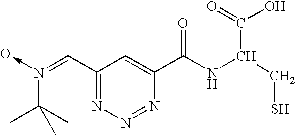

2-{[4-(tert-Butylnitrone)-[1,3,5]triazine-2-carbonyl]-amino}-3-mercapto-propionic acid

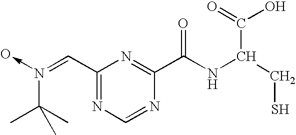

2-{[7-(tert-Butylnitrone)-pyrene-1-carbonyl]-amino}-3-mercapto-propionic acid

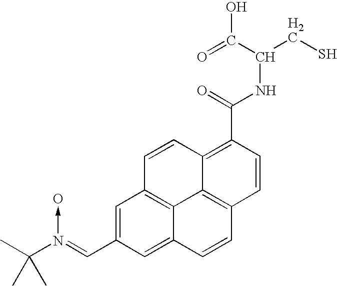

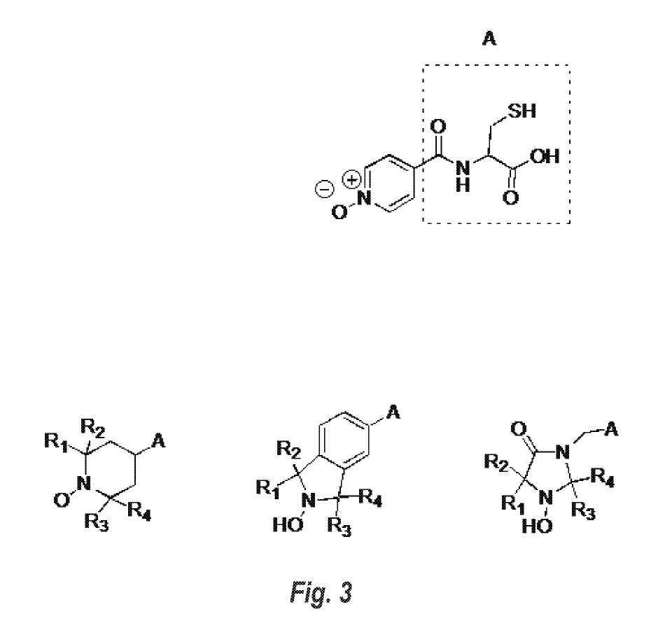

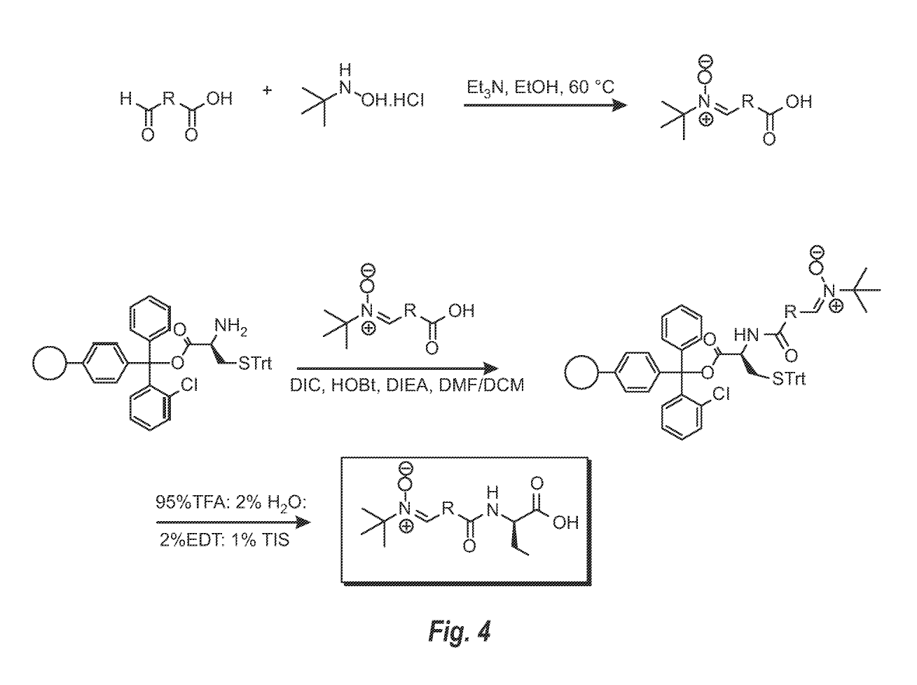

FIG. 3 shows additional antioxidant substituents, such as 4-(1-carboxy-2-mercaptoethylcarbamoyl)pyridine-1-oxide), and where “A” is as shown with the molecule having: 4-A-piperidinyl-1-oxyl; A-isoindoline, or 3-A-methyl-oximidazolidine-1-oxyl. As shown in FIG. 3, R1-R4 can be as described herein or any alkyl, such as methyl, ethyl, propyl, butyl, t-butyl, or the like. FIG. 4 shows that the antioxidant can be (E)-N-(4-(1-(4-carboxyphenyl)-2-mercaptoethylcarbamoyl)benzylidene)-2-methylpropan-2-amine oxide. Accordingly, when the antioxidant substituent is a piperidinyl-1-oxyl, isoindoline, or 3-A-methyl-1-oximidazolidine-1-oxyl, the substituents R1-R4 as shown in FIG. 3 can be as described herein, or can be any substituent described herein such as aliphatics or aromatics.

Additionally, any features, arrangements, constituents, motifs, or the like shown in connection with one antioxidant can be included in another antioxidant, and vice versa.

### II. Compositions/Route of Administration

In another embodiment of the present invention, a composition of matter (e.g., pharmaceutical composition) may include the novel compounds and a pharmaceutical carrier, excipient or diluent. The composition may comprise a novel compound cysteine acyl nitrone compound, bicyclic aryl nitrone, or other novel antioxidant compound described herein. In addition, the composition may also comprise one or more compounds which are described below. For example, selenium can be administered with the novel antioxidant of the present invention in a therapy for an appropriate condition as described herein or for treatments in which selenium is traditionally employed.

The novel compounds can be used in methods for treating (administering a pharmacologically effective amount of the novel compounds of the present invention), inhibiting, and/or preventing the various conditions, diseases, and events that may produce an imbalance in the oxidant/antioxidant. The novel compounds may be used alone in various formulations, such as oral, inhaled, intravenous or transdermal/transmucosal. For example, the present invention may provide a suitable formulation, administration mechanism, and/or dosage regimen for treating one or more of the above described conditions, diseases and events. The various formulations which may be employed may include pharmacologically suitable catalysts, carriers, excipients, diluents, reagents, and other such components for formulation as are known by those skilled in the art which may assist in effective and efficient delivery of the compound to a desired location.

The novel compounds can be used in a composition alone or in a combination for ameliorating the effects of exposure to a bacterial pathogen caused by natural causes/bioterror weapon, such as Bacillus Anthracis, including a composition of matter, at least one therapeutic dose of cysteine nitrone and a therapeutic dose of at least one antibiotic to said bacterial pathogen caused by natural causes/bioterror weapon. The invention may be used alone or in combination with a therapeutic dose of Selenium. A combination for prophylaxis of an impending bacterial pathogen caused by natural causes/bioterror weapon including a composition of matter, a therapeutic dose of cysteine nitrone and a prophylactic dose of at least one general antibiotic to said impending bacterial pathogen caused by natural causes/bioterror weapon. The invention may be used alone or in combination with a therapeutic dose of Selenium. The combination further including said at least one general antibiotic being selected from the group of Penicillins such as Penicillin, Ampicillin, Nafcillin, or Piperacillin, Beta-lactamase inhibitors such as Aztreonam, Imipenem, Ampicillin/sulbactam (Augmentum) or Pipercillin/tazobactam, or a subgroup including Beta-lactams such as Ceftriaxone, Cefuroxime, Quinolones such as Ciprofloxacin, Ofloxacin, Gatifloxacin, Sparfloxacin or Trovafloxacin, Tetracyclines such as Tetracycline, Doxycycline, or Minocycline, and Macrolides such as Erythromycin, Clarithromycin, or Azithromycin. The invention may be used alone or in combination with a therapeutic dose of Selenium.

The novel compounds can be used in a composition alone or in a combination for ameliorating the effects of exposure to a viral pathogen caused by natural causes/bioterror weapon, such as smallpox, including a composition of matter in an acceptable carrier, a therapeutic dose of cysteine nitrone and a therapeutic dose of at least one antiviral agent to said viral pathogen caused by natural causes/bioterror weapon. The invention may be used alone or in combination with a therapeutic dose of Selenium. A combination for prophylaxis of an impending viral pathogen caused by natural causes/bioterror weapon including a composition of matter in an acceptable carrier, a therapeutic dose of cysteine nitrone and a prophylactic dose of at least one general antiviral agent to said impending viral pathogen caused by natural causes/bioterror weapon. The combination further including said at least one general antiviral agent being selected from the group of cidofovir, Broad spectrum antivirals such as the Triazole carboxamine, Ribavirin, protease inhibitors such as Saquinavir, Ritonavir, Indinavir, and Nelfinavir, tricyclic amines such as Amantadine and Rimantadine, Meuraminic acid mimetics such as Relenza and Tamiflu, small cyclics such as Pleconaril, Nucleoside analogues such as Vidarabine, Acyclovir, Gancyclovir, valganciclovir, Nucleoside Reverse transcriptase inhibitors such as Zidovudine (AZT), Didanosine (ddI), Zaocaitabine (ddC), Stravudine (d4T), and Lamivudine (3TC), and Non-Nucleoside Reverse transcriptase inhibitors such as Nevirapine and Delaviridine.

In an embodiment, the novel compounds may be used alone or in combination with a therapeutic dose of Selenium.

The novel compounds can be used in a composition alone or in a combination for ameliorating the side effects of a vaccine against a bioterror weapon or disease more generally, including a composition of matter in an acceptable carrier, said vaccine, and a therapeutic dose of cysteine nitrone. The combination, further including said therapeutic dose of cysteine nitrone being selected from the group of water soluble cysteine nitrone compounds.

The novel compounds can be used in a composition alone or in a combination for prophylaxis of an impending potential combined viral and bacterial bioterror weapon including a composition of matter in an acceptable carrier, a therapeutic dose of a cysteine nitrone, a prophylactic dose of at least one general antiviral agent, and a prophylactic dose of at least one general antibiotic. The combination further including said at least one general antiviral agent being selected from the group of cidofovir, Broad spectrum antivirals such as the Triazole carboxamine, Ribavirin, protease inhibitors such as Saquinavir, Ritonavir, Indinavir, and Nelfinavir, tricyclic amines such as Amantadine and Rimantadine, Meuraminic acid mimetics such as Relenza and Tamiflu, small cyclics such as Pleconaril, Nucleoside analogues such as Vidarabine, Acyclovir, Gancyclovir, valganciclovir, Nucleoside Reverse transcriptase inhibitors such as Zidovudine (AZT), Didanosine (ddI), Zaocaitabine (ddC), Stravudine (d4T), and Lamivudine (3TC), and Non-Nucleoside Reverse transcriptase inhibitors such as Nevirapine and Delaviridine; and said at least Mone general antibiotic being selected from the group of Penicillins such as Penicillin, Ampicillin, Nafcillin, or Piperacillin, Beta-lactamase inhibitors such as Aztreonam, Imipenem, Ampicillin/sulbactam (Augmentum) or Pipercillin/tazobactam, or a subgroup including Beta-lactams such as Ceftriaxone, Cefuroxime, Quinolones such as Ciprofloxacin, Ofloxacin, Gatifloxacin or Trovafloxacin, Tetracyclines such as Tetracycline, Doxycycline, or Minocycline, and Macrolides such as Erythromycin, Clarithromycin, or Azithromycin. The invention

The compounds of the present invention may be variously formulated which may include a basic and acidic formulation. When the compounds of the invention are basic in nature, they may be capable of forming a wide variety of different salts with various inorganic and organic acids. Although such salts must be acceptable for administration to animals, it is often desirable in practice to initially isolate a compound from the reaction mixture as an unacceptable salt and then simply convert the latter back to the free base compound by treatment with an alkaline reagent, and subsequently convert the free base to an acceptable acid addition salt. The acid addition salts of the base compounds of this invention may be readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt may be obtained. When the compounds of the invention are acidic, acceptable acid addition salts of the base compounds of this invention will be used to form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts.

In an embodiment, the present invention includes the compounds present in a pharmaceutical preparation. The pharmaceutical preparations of the invention may include various amounts of the compounds and various diluents, excipients, carriers, and the like for assisting in the effective delivery and administration of the novel compound. Such preparations may routinely contain salts, buffering agents, preservatives, compatible carriers, and optionally other therapeutic agents. The salts may be acceptable, but non-acceptable salts may be conveniently used to prepare acceptable salts thereof and are not excluded from the scope of the invention. Acceptable salts include, but are not limited to, those prepared from the following acids: hydrochloric, hydrobromic, sulfuric, nitric, phosphoric, maleic, acetic, salicylic, citric, formic, malonic, succinic, hydriodic, tartaric, lactic, fumaric, oxalic, and the like. Also, acceptable salts may be prepared as alkaline metal or alkaline earth salts, such as sodium, potassium or calcium salts. As used herein, a composition of the compounds described above includes salts thereof.

The compounds of the invention may be combined with an acceptable carrier. The term “acceptable carrier” as used herein may include one or more compatible solid or liquid filler, diluents or encapsulating substances which may be suitable for administration into a mammal, such as a human, or other animal. The term “carrier” denotes an organic or inorganic ingredient, natural or synthetic, with which the active ingredient is combined to facilitate the application. The components of these carriers and/or combined compounds/carriers may be capable of being co-mingled with the molecules of the present invention, and with each other, in a manner such that there is no interaction which would substantially impair the efficacy and delivery performance of such combined compounds.

The various compositions described throughout the instant application may contain suitable buffering agents, including: acetic acid in a salt; citric acid in a salt; boric acid in a salt; and phosphoric acid in a salt. The various compositions may also contain suitable preservatives, such as: benzalkonium chloride; chlorobutanol; parabens and thimerosal.

The various compositions of the present invention may be formulated for various administration modes. Carrier formulations suitable for oral, subcutaneous, intravenous, intramuscular, and the like are contemplated. Administrations are described in more detail below and also may be found in Remington, The Science and Practice of Pharmacy, Nineteenth Edition, Mack Publishing Company, Eaton, Pa. (1995) which is hereby incorporated by reference.

When administered the compounds of the invention are administered using a variety of administration routes, whether the compounds are administered per se or in combination with various other components, such as acceptable carriers. The particular mode of administration selected may depend upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration may include oral, rectal, topical, buccal, nasal, bronchial, interdermal, vaginal or parenteral routes. Other modes of administration as may be contemplated by those of ordinary skill in the art may be employed without departing from the scope and spirit of the present invention. The term “parenteral” includes subcutaneous, intravenous, intraperitoneal, intramuscular, or infusion. Intravenous, intraperitoneal or intramuscular routes are not particularly suitable for long-term therapy and prophylaxis. They may, however, be preferred in emergency situations. Oral administration may be preferred for prophylactic administration because of the convenience to the subject as well as the dosing schedule.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The preparation to be administered may be in unit dosage form. In such form, the preparation may be subdivided into unit doses containing an appropriate quantity of the active component. The unit dosage form may be a packaged preparation, the package containing discrete quantities of preparation, such as packaged tablets, capsules, and powders in vials or ampoules. Also, the unit dosage form may be a capsule, tablet, cachet, or lozenge itself, or it may be the appropriate number of any of these in packaged form.

Compositions suitable for oral administration may be presented as discrete units, such as capsules, tablets, lozenges, pills, liquids, cachets, dragees, gels, lactose maize starch or derivatives thereof, talc, stearic acid, or its salts and the like each containing a predetermined amount of the active compounds. Other compositions include suspensions in aqueous liquids or non-aqueous liquids such as a syrup, elixir or an emulsion.

A tablet is a solid pharmaceutical dosage form containing a drug substance prepared either by compression or molding methods. The tablet may optionally include suitable diluents, additives, or excipients. Tablets may be of any shape or size which can be orally ingested. For instance, tablets may be discoid, round, oval, oblong, cylindrical, or triangular.

A gelatin capsule is a solid dosage form having a drug substance enclosed either in a hard or soft soluble container or shell made of a suitable form of gelatin. The hard gelatin capsule which is also referred to as the dry-filled capsule consists of two sections, one of which slips over the other to completely surround the drug formulation. Soft gelatin capsules include glycerin, sorbitol or a similar polyol for producing a soft globular gelatin shell which may be filled with a drug.

Lozenges also known as pastilles or troches are generally discoid shaped solid materials which contain the drug and a suitably flavored base. Lozenges are placed in the oral cavity and allowed to slowly dissolve in order to deliver the drug.

Cachets are a capsule like container having a drug encapsulated in a shell. The drug is placed into the two halves of the shell and the edges of the two halves are sealed to produce a sealed capsule like container.

Dragees are a coated form of tablet. The dragee core is coated with flavoring solution such as concentrated sugar solutions which may optionally contain gum arabic, talc, polyvinyl, pyrrolidone, carbopol, gel, polyethylene glycol, and/or titanium dioxide, lacquer solutions, and suitable organic solvents or solvent mixtures. The dragee coatings may also contain dyestuffs or pigments.

Compositions suitable for rectal and vaginal administration include suppository bases and retention enemas which include conventional suppository bases such as cocoa butter or other glycerides, natural or hardened oils, waxes, fats, semi-liquid polyols and the like.

Compositions suitable for parenteral administration conveniently comprise a sterile aqueous preparation of the compounds of the invention, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables.

Compositions suitable for buccal administration may take the form of tablets, lozenges, and the like.

Topical formulations include the drug of interest in a cream, lotion, gel, transdermal patch or powdered form. Optionally the topical formulations may include permeation enhancers that are useful for enhancing the uptake of the drug through the skin. Permeation enhancers are well known in the art and are commercially available.

Compositions suitable for nasal or broncho-pulmonary administration include emulsions for aerosol delivery. In general an aerosol is a finely dispersed mist, foam, or semisolid material which is delivered to the nose or mouth as a spray powered by a liquefied or compressed gas or a pump. Aerosols sprays may be delivered in a pressurized pack or nebulizer with the use of a propellant, such as fluorocarbon propellants (e.g., dichlorodifluoromethane, trichlorofluoromethane, dichlorotetrafluoroethane), hydrocarbon propellants or compressed gases such as nitrogen, nitrous oxide, and carbon dioxide. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

For topical ocular administration, direct application to the affected eye may be employed in the form of a formulation as eyedrops, aerosol, gels or ointments, or may be incorporated into collagen (such as poly-2-hydroxyethylmethacrylate and co-polymers thereof), or a hydrophilic polymer shield. The materials may also be applied as a contact lens or via a local reservoir or as a subconjunctival formulation.

Other delivery systems may include time-release, delayed release or sustained release delivery systems. Such systems may avoid repeated administrations of the compounds described above, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109, which is herein incorporated by reference. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the compound of the invention is contained in a form within a matrix such as those described in U.S. Pat. Nos. 4,452,775, 4,667,014, 4,748,034 and 5,239,660 (herein incorporated by reference) and (b) difusional systems in which an active component permeates at a controlled rate from a polymer such as described in U.S. Pat. Nos. 3,832,253, and 3,854,480 (herein incorporated by reference). In addition, pump-based hardware delivery systems may be used, some of which are adapted for implantation.

In an embodiment, a kit can be provided for the administration of one or more of the novel compounds to a subject experiencing a disorder or condition, such as those described herein. The kit may include a package housing a container containing the compounds of the invention in an amount effective to exert a pharmacological effect and an acceptable carrier, and instructions for using the compound to treat the subject experiencing the acute disorder.

The routes of administration can be relative to a subject, a female that is pregnant, or a fetus.

### IV. Methods of Use

In one embodiment, the compounds of the invention are presented as potential therapeutic agents for indications that have been reported to be amenable to antioxidant treatment or that involve free-radical generation including, but not limited to: cerebrovascular stroke, myocardial infarction and dysfunction, retinal ischemia and damage including macular degeneration and other degenerative disorders of the retina, renal ischemia, arteriosclerosis and other cardiovascular diseases, amyotrophic lateral sclerosis, Parkinson's disease, Alzheimer's disease, Huntington's disease, multiple sclerosis, head trauma and traumatic brain injury, nerve injury and neuropathies, migraine, schizophrenia and other disorders of cognition, mood disorders and other disorders of affect, pancreatitis and other pancreatic disorders, the treatment of diabetes and related complications, epilepsy, transplant and graft failure or rejection, hepatitis and jaundice-induced liver disorders, lung injury and damage, gastric ulcer, endotoxemia, aging and senescence, preterm labor, fetal damage due to intrauterine hyoxemia or ischemia, the treatment and prophylaxis of pain syndromes (acute and chronic or neuropathic), arthritis and other autoimmune disorders, asthma and allergic reactions, inflammatory bowel disease, irritable bowel syndrome, uveitis, cancer, the treatment of complications and disorders arising from cancer therapy, and alopecia (hair loss). Examples of conditions that can be treated, inhibited, and/or prevented with the novel compounds can include preterm labor, fetal inflammatory response syndrome and its complications, arthritis, Parkinson's disease, Alzheimer's disease, stroke, uveitis, asthma, myocardial infarction, traumatic brain injury, spinal cord injury, neurodegenerative disorders, alopecia (hair loss), inflammatory bowel disease, autoimmune disorders, renal disorders, obesity, eating disorders, cancer, schizophrenia, epilepsy, sleeping disorders, cognition, depression, anxiety, high blood pressure, lipid disorders and atherosclerosis, or the like.

A method of administering a pharmacologically effective amount of the compound of the present invention is a preferred embodiment of the present invention. The method may be for treating a mammal susceptible to or afflicted with a condition associated with excessive oxidation, such as, for example arthritis, uveitis, asthma, myocardial infarction, traumatic brain injury, acute spinal cord injury, alopecia (hair loss), inflammatory bowel disease or autoimmune disorders. The method may include administering an effective amount of the antioxidant compound, wherein the compound is formulated as one or more of the compositions of matter previously described.

The method may be for treating a mammal susceptible to or afflicted with a condition that gives rise to pain responses or that relates to imbalances in the maintenance of basal activity of sensory nerves. The antioxidant compounds have use as analgesics for the treatment of pain of various geneses or etiology, for example, acute, inflammatory pain (such as pain associated with osteoarthritis and rheumatoid arthritis); various neuropathic pain syndromes (such as post-herpetic neuralgia, trigeminal neuralgia, reflex sympathetic dystrophy, diabetic neuropathy, Guillian Barre syndrome, fibromyalgia, phantom limb pain, post-masectomy pain, peripheral neuropathy, HIV neuropathy, and chemotherapy-induced and other iatrogenic neuropathies); visceral pain (such as that associated with gastroesophageal reflex disease, irritable bowel syndrome, inflammatory bowel disease, pancreatitis, and various gynecological and urological disorders); dental pain; and headache (such as migraine, cluster headache and tension headache).

In an embodiment, the method may be for treating a mammal susceptible to or afflicted with neurodegenerative diseases and disorders such as, Parkinson's disease, Alzheimer's disease and multiple sclerosis; diseases and disorders which are mediated by or result in neuroinflammation such as, traumatic brain injury, stroke and encephalitis; centrally-mediated neuropsychiatric diseases and disorders such as, depression, mania, bipolar disease, anxiety and schizophrenia; eating disorders, sleep disorders and cognition disorders; epilepsy and seizure disorders; prostate, bladder and bowel dysfunction such as, urinary incontinence, urinary hesitancy, rectal hypersensitivity, fecal incontinence, benign prostatic hypertrophy and inflammatory bowel disease; respiratory and airway diseases and disorders such as, allergic rhinitis, asthma, reactive airway diseases and chronic obstructive pulmonary disease; diseases and disorders which are mediated by or result in inflammation such as, rheumatoid arthritis, osteoarthritis, myocardial infarction, various autoimmune diseases and disorders, uveitis and atherosclerosis; itch/pruritus such as, psoriasis; alopecia (hair loss); obesity; lipid disorders; cancer; high blood pressure; spinal cord injury; and renal disorders. The method includes administering an effective condition-treating or condition-preventing amount of the novel antioxidant compound, which may be variously formulated, such as into one or more of the compositions previously described.

In one embodiment, the novel antioxidant compounds can be capable of modifying mammalian inflammatory pathways. For instance, novel compounds of the present invention may include substituted N-aryl, heteroaromatic or bicyclic aryl cysteine nitrones, or derivatives thereof with antioxidant substituents as active ingredients. These antioxidant compounds may assist in the treatment, prevention or amelioration of a range of conditions in mammals such as, but not limited to, pain of various genesis or etiology, for example, preterm labor, the fetal inflammatory syndrome, acute, chronic, inflammatory and neuropathic pain, dental pain and headache (such as migraine, cluster headache and tension headache).

Additionally, the novel antioxidant compounds may up-regulate and/or down-regulate one or more specific metabolic pathways within various cellular response mechanisms, such as an inflammatory pathway, in other exemplary embodiments. The novel compounds of the present invention may provide a mechanism and method for regulating the presence or absence of other antioxidant(s) within specific environments, such as cellular environments.

For some disorders the novel antioxidant compounds can be used alone, or combined together, or in combination with other agents such as antibiotic and/or antivirals to ameliorate the toxic effects of ROIs, which may be caused by ischemic events, drug toxicity, radiation exposure or due to infection either naturally acquired or after Bioterror incidents such as Bacillus anthracis and smallpox virus during testing or vaccination. The novel compounds may be utilized for treatment prior to or in combination with treatment with antibiotic or antiviral therapy (antiviral agents). It is contemplated that such use may assist in ameliorating the toxic effects of ROIs brought about by various causes, such as exposure and/or infection with various viral organisms, or alternatively, after radiation exposure.

A method of ameliorating the effects of exposure to a bacterial pathogen caused by natural causes/bioterror weapon such as Bacillus Anthracis including the following steps: administering in a composition of matter in an acceptable carrier at least one therapeutic dose of an antioxidant (e.g., cysteine nitrone or derivative); and administering at least one therapeutic dose of at least one antibiotic to said bacterial bioterror weapon. A method of prophylaxis of impending exposure to a bacterial pathogen caused by natural causes/bioterror weapon such as Bacillus Anthracis including the following steps: administering in a composition of matter in an acceptable carrier a course of therapeutic doses of an antioxidant; and administering a course of prophylactic doses of at least one antibiotic to said bacterial pathogen caused by natural causes/bioterror weapon. The method further including selecting said at least one general antibiotic from the group of Penicillins such as Penicillin, Ampicillin, Nafcillin, or Piperacillin, Beta-lactamase inhibitors such as Aztreonam, Imipenem, Ampicillin/sulbactam (Augmentum) or Pipercillin/tazobactam, or a subgroup including Beta-lactams such as Ceftriaxone, Cefuroxime, Quinolones such as Ciprofloxacin, Ofloxacin, Gatifloxacin, Sparfloxacin or Trovafloxacin, Tetracyclines such as Tetracycline, Doxycycline, or Minocycline, and Macrolides such as Erythromycin, Clarithromycin, or Azithromycin.

A method of ameliorating the effects of exposure to a viral pathogen caused by natural causes/bioterror weapon such as smallpox including the following steps: administering in a composition of matter in an acceptable carrier at least one therapeutic dose of an antioxidant (e.g., cysteine nitrone or derivative); and administering at least one therapeutic dose of at least one antiviral agent to said viral bioterror weapon. A method of prophylaxis of impending exposure to a viral pathogen caused by natural causes/bioterror weapon such as smallpox including the following steps: administering in a composition of matter in an acceptable carrier a course of therapeutic doses of an antioxidant; and administering a course of prophylactic doses of at least one general antiviral agent to said viral pathogen caused by natural causes/bioterror weapon. The method further including selecting said at least one general antiviral agent from the group of cidofovir, Broad spectrum antivirals such as the Triazole carboxamine, Ribavirin, protease inhibitors such as Saquinavir, Ritonavir, Indinavir, and Nelfinavir, tricyclic amines such as Amantadine and Rimantadine, Meuraminic acid mimetics such as Relenza and Tamiflu, small cyclics such as Pleconaril, Nucleoside analogues such as Vidarabine, Acyclovir, Gancyclovir, valganciclovir, Nucleoside Reverse transcriptase inibiitors such as Zidovudine (AZT), Didanosine (ddI), Zaocaitabine (ddC), Stravudine (d4T), and Lamivudine (3TC), and Non-Nucleoside Reverse transcriptase inhibitors such as Nevirapine and Delaviridine.

A method of ameliorating the side effects of a vaccine against a bioterror weapon or disease more generally, including the following steps: administering in a composition of matter in an acceptable carrier a course of therapeutic doses of an antioxidant prior to administration of said vaccine; administering said vaccine; continuing said administration of a course of therapeutic does of antioxidant subsequent to administration of said vaccine at least until any potential side effects of said vaccine are attenuated.

Another method of ameliorating the side effects of a vaccine against a bioterror weapon or disease more generally, including the following steps: administering said vaccine; administering in a composition of matter in an acceptable carrier a therapeutic dose of antioxidant contemporaneously to administration of said vaccine. The method further including the additional step: continuing said administration of a course of therapeutic doses of antioxidant subsequent to administration of said vaccine at least until any potential side effects of said vaccine are attenuated. The method further including said therapeutic dose of antioxidant being selected from the group of water soluble antioxidant compounds.

A method of prophylaxis of impending exposure to an impending potential combined viral and bacterial pathogen caused by natural causes/bioterror weapon including the following steps: administering in a composition of matter in an acceptable carrier a course of therapeutic doses of antioxidant; and administering a course of prophylactic doses of at least one general antibiotic; and administering a course of prophylactic doses of at least one general antiviral agent. The method further including selecting said at least one general antiviral agent from the group of cidofovir, Broad spectrum antivirals such as the Triazole carboxamine, Ribavirin, protease inhibitors such as Saquinavir, Ritonavir, Indinavir, and Nelfinavir, tricyclic amines such as Amantadine and Rimantadine, Meuraminic acid mimetics such as Relenza and Tamiflu, small cyclics such as Pleconaril, Nucleoside analogues such as Vidarabine, Acyclovir, Gancyclovir, valganciclovir, Nucleoside Reverse transcriptase inhibitors such as Zidovudine (AZT), Didanosine (ddI), Zaocaitabine (ddC), Stravudine (d4T), and Lamivudine (3TC), and Non-Nucleoside Reverse transcriptase inhibitors such as Nevirapine and Delaviridine; and selecting at least one general antibiotic from the group of Penicillins such as Penicillin, Ampicillin, Nafcillin, or Piperacillin, Beta-lactamase inhibitors such as Aztreonam, Imipenem, Ampicillin/sulbactam (Augmentum) or Pipercillin/tazobactam, or a subgroup including Beta-lactams such as Ceftriaxone, Cefuroxime, Quinolones such as Ciprofloxacin, Ofloxacin, Gatifloxacin, Sparfloxacin or Trovafloxacin, Tetracyclines such as Tetracycline, Doxycycline, or Minocycline, and Macrolides such as Erythromycin, Clarithromycin, or Azithromycin.

A method of ameliorating radiation damage, can include the step of administering a therapeutic dose of antioxidant.

It is to be understood that the compositions, administration methods, and indications for efficacy of the novel compound of the present invention, whether alone or in combination with any of the various other components (i.e., antibiotic, anti-viral agent, vaccine, and the like) may be utilized for all potential indications identified throughout the specification of the instant application.

In one embodiment, the novel compounds can be used in any therapy that utilizes the anti-oxidant characteristics of the compounds. This can include using the novel compounds for treating, inhibiting, and/or preventing any of the following: abnormal intrauterine conditions; newborn brain abnormalities; intrauterine oxygen deficiency, either chronic or acute; perinatal brain damage; acute oxygen depravation; perinatal hypoxic ischemic reperfusion injury; brain damage associated with acute oxygen depravation; conditions associated with primary cell death due to lack of oxygen; conditions associated with secondary cell death due to inflammatory response by reperfusion with oxygenated blood; acute ischemic reperfusion during labor; cerebral palsy; intrauterine growth restriction (IUGR); chronic fetal hypoxemia; fetal inflammatory response in the brain; preterm labor and/or birth associated with fetal inflammatory response; idiopathic preterm birth; or other condition caused by excessive oxidation.

The ability to treat, inhibit, and/or prevent intrauterine conditions arises from many of the foregoing conditions being associated with excess free oxygen radicals. Thus, antioxidants of the invention can be used in the corresponding therapy. The antioxidant can be administered to the mother or directly to the fetus.

In one embodiment, a method of treating an unborn subject can include identifying the unborn subject is at risk for or has acute or chronic oxygen depravation.

In one embodiment, a method of treating an unborn subject can include detecting a brain abnormality in the unborn subject.

In one embodiment, a method of treating an unborn subject can include detecting an abnormal intrauterine environment. This can include acute or chronic oxygen depravation, perinatal hypoxic ischemic reperfusion, hostile intrauterine environment, excess oxygen free radicals, or the like.

In one embodiment, glutathione can be used as an additional antioxidant capable of scavenging a wide range of oxygen free radicals. Also, additional antioxidants can include ascorbic acid and vitamin E, which scavenge a limited number of oxygen free radicals types. Additionally, the additional antioxidant can be N-acetyl cysteine (NAC), which is a glutathione precursor used in humans for the treatment of acetaminophen toxicity and lung disease in patients with cystic fibrosis.

In one embodiment, the present invention can include a method of treating, inhibiting, and/or preventing fetal hypoxia by administering an antioxidant. Chronic hypoxemia can trigger an inflammatory response in the fetal brain at similar locations as the abnormalities in children with cerebral palsy. Also, chronic hypoxia can lead to a loss of fetal neurons due to excess oxygen free radical generation. The initiating step in the generation of hypoxia mediated brain inflammation is the induction of an enzyme known as inducible nitric oxide synthase (iNOS). The terminal step in the damage producing pathway is the generation of excess oxygen free radicals. Inhibition of iNOS prevents the fetal brain inflammatory response and the associated loss of neurons despite continued hypoxia. However, iNOS is heavily expressed in the placenta; its function is unknown. The provision of increased oxygen free radical scavenging capacity with an antioxidant of the present invention can prevent the fetal brain inflammatory response and the associated loss of neurons despite continued hypoxia, but does not interfere with the production of iNOS. Contrary to conventional wisdom, fetal brain damage secondary to chronic oxygen depravation is not directly caused by the low oxygen level, but rather by the fetal adaptation to low oxygen. It is possible to prevent fetal brain damage secondary to chronic oxygen depravation without delivering the fetus who may be extremely premature; administer an antioxidant of the present invention indirectly by providing to mother, or directly by injection into the fetus. Thus, the antioxidants of the present invention can be used to treat, inhibit, or prevent any of the conditions or symptoms described with regard to fetal hypoxia.

In one embodiment, the present invention can include a method of treating, inhibiting, and/or preventing idiopathic, spontaneous preterm birth. Biomarkers in the amniotic fluid environment can strongly predict preterm birth in women displaying symptoms of preterm labor. These markers can be used to identify women with the syndrome of dysfunctional cervical ripening. However, women who ultimately experience idiopathic preterm birth have no such biomarkers present at 16 weeks of pregnancy. Inflammation mediated preterm birth whether associated with dysfunctional cervical ripening or idiopathic preterm labor is acquired over the course of pregnancy. It is possible to identify at risk women for preterm birth so that an antioxidant can be administered to the women. It is also possible to identify and treat women at risk with an antioxidant such as those described herein to further reduce the likelihood of idiopathic preterm birth and the fetal inflammatory response syndrome.

### V. Synthesis

Experimental procedures: Unless otherwise stated, reagents were purchased from commercial suppliers and are used without further purification. The ¹H NMR spectra were recorded at 400 MHz on a Bruker Avance-400 spectrometer using CDCl₃ or MeOD as solvent, δ values in ppm (TMS as internal standard), and J (Hz) assignments of ¹H resonance coupling.

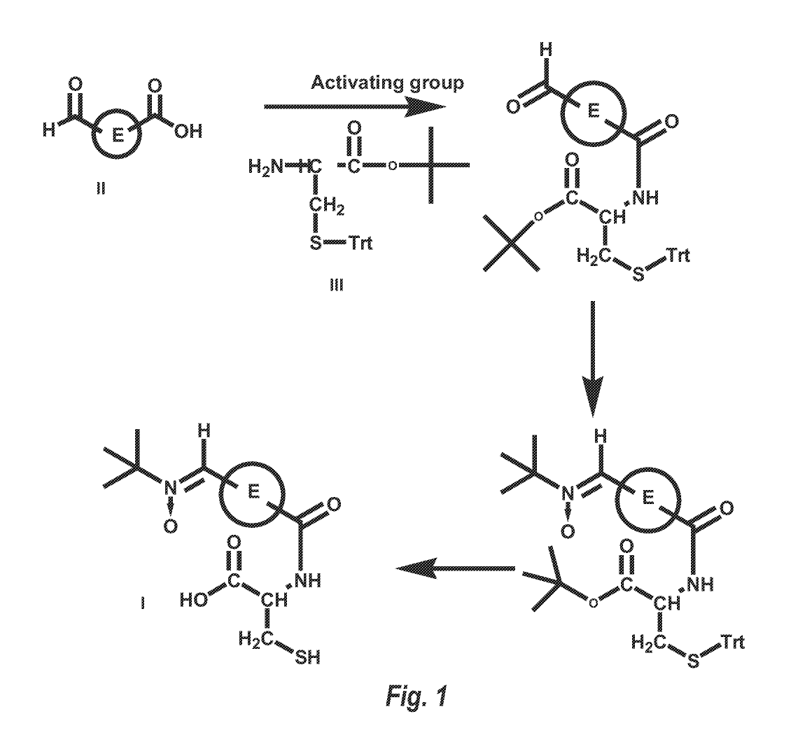

In an embodiment, the present invention provides a process for the preparation of a compound of general Formula I as shown in FIG. 1. In FIG. 1, the molecular formulas are shown by I, II, and III, which are Formula I, Formula II, and Formula III. This process involves reaction of an aldehyde of general Formula II with N-tert-butylhydroxylammonium acetate (Formula III). In an alternative embodiment, the present invention provides a method for preparing and recovering a compound of general Formula I. In the first step of this process, the compound is prepared as described below. In a subsequent step, the compound is isolated. In Formula I, E can be any group as described herein.

In additional aspects, this invention provides methods for synthesizing the aryl, heteroaromatic and bicyclic aryl cysteine nitrone compounds or their derivatives, which are antioxidants.

In the process of FIG. 1, an aldehyde of general Formula II is reacted with N-terbutylhydroxylammonium acetate to form 2-[n-(tert-Butylnitrone)-acylamino]-3-mercapto-propionic acid. The E of Formula II can have a ring substituted at any suitable position in the ring as described herein with respect to the general group E. The compounds of general Formula I and Formula II may be acids or they may be salts. The salts of compounds of Formula I and Formula II will normally be those formed with pharmaceutically acceptable cations. The cation may be a monovalent material such as sodium, potassium, lithium, ammonium, alkylammonium or diethanolammonium. Alternatively, it may be a polyvalent cation such as calcium, magnesium, aluminium or zinc. It may also be a mixed salt formed with a polyvalent cation such as calcium or magnesium in combination with a pharmaceutically acceptable anion such as halide (for example chloride), phosphate, sulfate, acetate, citrate or tartrate.

The aldehydes of general Formula II are either commercially available or may be prepared from commercially available materials using methods that are well known in the art. Commercial n-formyl-1-arylcarboxylic acid where n=2, 3, 4, 5, 6, 7, 8 or any suitable position in the aromatic ring can contain small but significant amounts of the corresponding aryl alcohol and of sodium chloride as impurities. It is preferable, but not essential, that such material is purified before use in the process of the present invention. n-Formyl-1-arylcarboxylic acid is typically associated with varying amounts of water. The proportion of such water generally is not critical to the process of the present invention but generally may be taken into account when determining the overall composition of the Formula I forming reaction mixture.

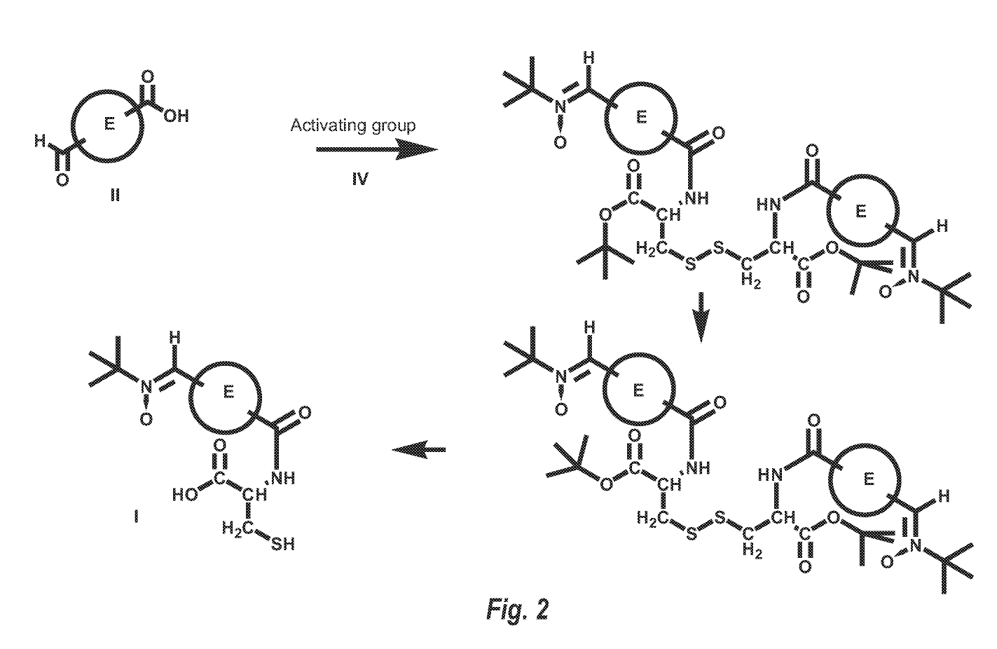

FIG. 2 shows a method of e synthesis can be carried out in three to four steps or more. The first step involves the activation of the carboxylic acid group. The second step is the condensation step with L-(trityl) cysteine tert-butyl ester (III) or cysteine tert-butyl ester (IV). Both L-(trityl) cysteine tert-butyl ester (III) or cysteine tert-butyl ester (IV) is either commercially available or may be prepared from commercially available materials using methods that are well known in the art. The third step is the condensation with the N-terbutylhydroxylammonium acetate and the final step is the deprotection. This reaction is typically conducted in a batch mode with agitation. It is contemplated that the synthesis of the novel compound of the present invention may be carried out continuously in a flow reaction system.

The activation of the carboxylic acid is carried out by converting the acid to the corresponding acyl halide where halides include chloride, fluoride, bromide. Further they may be converted to acyl azide, acyl imidazole, anhydrides or esters formation.

### Method A

The formation of the acid chloride is carried out using a suitable reagent like thionyl chloride, oxalyl chloride, phosphorus trichloride, phosphorus oxychloride, and phosphorus pentachloride. In these processes it is preferred that in general about 1.0 to 10 equivalents of reagent for every equivalent of the acid. In these processes it is preferred to add a catalytic amount of N,N-dimethylformamide. In case of acid sensitive substrates, cyanuric chloride (2,4,6-trichloro-1,3,5-triazine). The formation of the acyl fluoride is carried out using a suitable reagent like cyanuric fluoride, N,N-tetramethylfluoroformamidinium hexa fluorophosphates (TFFH), Diethylaminosulfur trifluoride (DAST), and deoxofluor. The formation of the acyl bromide is carried out using a suitable reagent like a-bromoacetyl bromide, thionyl bromide, triphenyl phosphene/N bromosuccinimide, triphenylphosphenelbromine, boron tribromide/aluminium oxide, (BrCO)₂, 1-bromo-N,N-2-trimethyl-1-propenylamine. It is preferred but not restricted to, a suitable chlorinated solvent such as chloroform, dichloromethane, chloroform, dichloroethane. The proportion of reaction solvent is typically maintained at about 2 to 25 mL of solvent per gram of reactant product or greater, with proportions of from 8 to 20 and especially 12 to 15 mL/g being preferred. The condensation is conducted at a temperature from about 0 C temperature to about 50 C, good results being achieved at temperatures of from about 1° C. to about 4° C., with temperatures of from about 15 C to about 25 C being preferred.

### Method B

The formation of the acyl azide is carried out using acid chloride and sodium azide. Further it is carried out using diphenylphosphonic azide. In these processes it is preferred that in general about 1.0 to 10 equivalents of reagent for every equivalent of the acid is used.

### Method C

The formation of the acylimidazoleis carried out using methyl ester and a suitable reagent like carbonyl diimidazole (CDI), N,N′-carbonylbis(3-methylimidazolinium triflate (CBMIT). It is preferred but not restricted to, a suitable solvent such as tetrahydrofuran (THF), 1,4-dioxane, 1,2-dichloroethane, dichloromethane, N,N-dimethyl formamide, acetonitrile, dimethylsulfoxide or a mixture of solvents in which the starting materials are sufficiently soluble. The proportion of reaction solvent is typically maintained at about 2 to 15 mL of solvent per gram of reactant product or greater, with proportions of from 3 to 12 and especially 5 to 8 mL/g being preferred. The condensation is conducted at a temperature from about 0° C. to about 60° C., good results being achieved at temperatures of from about 10° C. to about 40° C. The condensation of the present invention is carried out in using a suitable base. It is preferred but not restricted to, dimethylisopropyl amine (DIEA), trimethylamine (TEA), benzyl dimethylamine, N-methyl-2-pyrrilidone (NMP), N-methyl morpholine, pyridine, The use of DIEA is particularly preferred. In this process it is preferred that in general about 1 to 10 equivalents of base is used for each equivalent of the formyl arylcarboxylic acid (II). It is particularly preferred that about 2.5 to 5 equivalents of base is used. The condensation reaction is relatively facile and is typically essentially complete in from about 5 minutes to about 20 hours with reaction time of from 1 hour to 5 hours being typical. In practice, the degree of reaction is monitored analytically and the reaction is continued until a suitable degree of reaction is achieved.

### Method D

The formation of the anhydride carried out using ethylchloroformate, 2-ethoxy-1-ethoxycarbonyl-1,2-dihydroquinoline (EDDQ), Dicyclohexyl carbodiimide (DCC), diisopropyl carbodiimide (DIC) and 1-ethyl-3-(30-dimethylamino)carbodiimide HCl salt (EDC). The coupling reactions are further helped by the addition of nucleophiles like dimethylaminopyridine (DMAP).

### Method E

For the formation active esters are selected from, but not restricted to esters from alcohols like hydroxybenzotriazole (HOBt), p-nitrophenol (PNP), pentafluorophenol (PFP), 2,4,5-Trichlorophenol, N-hydroxy-5-norbornene-endo-2,3-dicarboxylmide (HONB), N-hydroxysuccinimide (HOSu), Hydroxy-7-azabenzotriazole (HOAt), carried out using DCC. The synthesis of succinimidyl esters is also carried out with N,N′-disuccinimidyl carbonate (DSC), and uranium salt like O—(N-succinimidyl)-N,N,N′,N′,-tetramethyluronium tetrafluoroborate (TSTU).

The choice of uranium salts is from Benzotriazolvyloxy-bis(pyrroltdino)-carbonium hexaflouorophosphate (BCC), O-(3,4-dihydro-4-oxo-1,2,3-benzotriazin-3-yl-1,1,3,3-tetramethyluronium tetrafluoroborate (TDBTU), 2-(5-norbornene-2,3-dicarboximido)-1,1,3,3-tetramethyluronium tetrafluoroborate (TNTU), O-(1,2-dihydro-2-oxo-1-pyridyl)-1,1,3,3-tetramethyluronium tetrafluoroborate TPTU, TSTU, O-(7-azabenzotriazol-1-yl)-1,1,3,3-bis(tetramethylene)-uronium hexafluorophosphate (HAPyU), 2-chloro-1,3-dimethylimidazolidinium hexafluorophosphate (CIP), Bis(tetramethylene)fluoroformamidinium Hexafluorophosphate (BTFFH), S-(1-oxido-2-pyridinyl)-1,1,3,3-tetramethylthiouronium hexafluorophosphate (HOTT), S-(1-oxido-2-pyridinyl)-1,1,3,3-tetramethylthiouronium tetrafluoroborate (TOTT).

The preferred route of activation of the present invention is carried out in using a suitable activating reagent like O—(N-succinimidyl)-N,N,N′,N′,-tetramethyluronium tetrafluoroborate (TSTU) to form the O—(N-succinimido) ester derivative. In this process it is preferred that in general about 1 to 3 equivalents of TSTU is used for each equivalent of the cysteine. It is particularly preferred that about 1.0 to 2.5 equivalents of TSTU is used.

In the second step, which is condensation of the present invention, it is preferred that in general about 1.0 to 2.5 equivalents of L-(trityl) cysteine tert butyl ester (III) is used for each equivalent of the formyl arylcarboxylic acid (II). It is particularly preferred that about 1.0 to 1.0 equivalents of cysteine methyl ester (III) is used. The condensation of the present invention is carried out in solution, using a suitable inert solvent in which the starting materials are sufficiently soluble. It is preferred but not restricted to, a suitable polar organic solvent such as N,N-dimethyl formamide (DMF), dimethylsulforxide (DMSO), 1,4-Dioxane,tetrahydrofuran (THF), water (H₂O) or a mixture of solvents in which the starting materials are sufficiently soluble.

It is further preferred that the reaction mixture contains a suitable percentage of water, generally less than 25% by volume, such as from about 2% to 25% by volume. It is particularly preferred that the solvent contains about 10% by volume of water. It has been found that the presence of a suitable amount of water provides significant advantages as it helps dissolve the reagent thereby enhancing the rate of the reaction.

The proportion of reaction solvent is typically maintained at about 2 to 15 mL of solvent per gram of reactant product or greater, with proportions of from 3 to 12 and especially 5 to 8 mL/g being preferred.

The condensation is conducted at a temperature from about 0 C to about 50 C, good results being achieved at temperatures of from about 1° C. to about 4° C., with temperatures of from about 15 C to about 25 C being preferred.

The condensation of the present invention is carried out using a suitable base, that may be selected from, but not restricted to, dimethylisopropyl amine (DIEA), trimethylamine (TEA), benzyl dimethylamine, N-methyl-2-pyrrilidone (NMP), dimethylamino pyridine (DMAP), N-methyl morpholine, pyridine, The use of DIEA is particularly preferred. In this process it is preferred that in general about 2 to 10 equivalents of base is used for each equivalent of the formyl arylcarboxylic acid (II). It is particularly preferred that about 2.5 to 5 equivalents of base is used.

The condensation reaction is relatively facile and is typically essentially complete in from about 5 minutes to about 5 hours with reaction time of from 30 to 90 minutes being typical. In practice, the degree of reaction is monitored analytically and the reaction is continued until a suitable degree of reaction is achieved.

The third step is the condensation of the N-tert-butylhydroxylammonium acetate (III) with the aldehyde (II) derived in the first condensation step. This reaction is typically conducted in a batch mode with agitation. It could, if desired, be carried out continuously in a flow reaction system.

In this process it is preferred that in general about 1.25 to 2.5 equivalents of N-terbutylhydroxylammonium acetate (III) is used for each equivalent of the aldehyde (II). It is particularly preferred that about 1.6 to 2.0 equivalentsof N-tert-butylhydroxylammonium acetate (III) is used.

The condensation of the present invention is carried out in solution, using a suitable inert solvent in which the starting materials are sufficiently soluble. It is preferred that a suitable polar organic solvent such as an alcohol, or mixture of alcohols, is used as solvent.

It is particularly preferred that the solvent is methanol. It is further preferred that the reaction mixture contains a suitable percentage of water, generally less than 10% by volume, such as from about 2% to 10% by volume. It is particularly preferred that the solvent contains about 5% by volume of water. It has been found that the presence of a suitable amount of water provides significant advantages, particularly with regards to inhibiting the conversion of the aldehyde (II) into the undesirable acetal side product by reaction with the solvent alcohol.

The presence of a suitable amount of water in the solvent also increases the solubility of the aldehyde starting material and thereby significantly improves the kinetics of the process and enables the use of a more concentrated system. The proportion of reaction solvent is typically maintained at about 2 to 15 mL of solvent per gram of nitrone product or greater, with proportions of from 3 to 10 mL/g and especially 5 to 8 mL/g being preferred. The condensation is conducted at a temperature from about ambient temperature to about 150 C, good results being achieved at temperatures of from about ambient to about 125 C, with temperatures of from about 40 C to about 100 C being preferred. The condensation reaction is relatively facile and is typically essentially complete in from about 15 minutes to about 5 hours with reaction time of from 30 to 90 minutes being typical. In practice, the degree of reaction is monitored analytically and the reaction is continued until a suitable degree of reaction is achieved.

The third step is the removal of the protecting groups and may be achieved by using standard techniques that are well known in the art. In this process it is preferred that large excess of marcaptans are used as a solvent, in general about >25 equivalents of ethanethiol with trifluoroacetic acid is used for each equivalent of the product. It is particularly preferred that about 25 ml of ethane thiol and 25 ml of trifluoroacitic acid for every 10 mmol of the compound. The deprotection is conducted at a temperature from about −10 C to about 40 C, good results being achieved at temperatures of from about 10 C to 25 C The deprotection reaction is relatively facile and is typically essentially complete in from about 1 hour to about 10 hours with reaction time of from 2 h to 6 h being typical. In practice, the degree of reaction is monitored analytically and the reaction is continued until a suitable degree of reaction is achieved.

The isolation of the product of Formula I formed in the above condensation may be achieved by using standard techniques that are well known in the art. It is particularly advantageous that the product be isolated using a suitable crystallisation technique. Thus in a typical isolation, on completion of the reaction of the aldehyde (II) with N-tert-butylhydroxylammonium acetate (III), the reaction mixture is cooled to ambient temperature and then filtered in order to remove any insoluble material. The filtrate is then adjusted to a temperature that may be from 0 C up to the reflux temperature of the solvent, but is preferably from 35 to 50 C, and crystallisation is induced by the addition of a suitable crystallisation agent such as isopropanol or ethyl acetate. The optimal precipitation temperature may vary depending on the scale of the reaction, on whether the suspension is stirred or allowed to stand, and on the desired particle size of the solid product.

The crystallisation agent is typically an organic liquid that is miscible with the reaction solvent but one in which the nitrone product is less soluble. The agent is also generally a volatile material, such as a material having 5 or less carbon atoms. The solid product is isolated by filtration and dried. The use of isopropanol, as a crystallisation agent, is particularly preferred.

Alternatively, crystallisation may be induced by the addition of a suitable agent such as isopropanol or ethyl acetate without the filtrate having first been heated. Again, the use of isopropanol is particularly preferred.

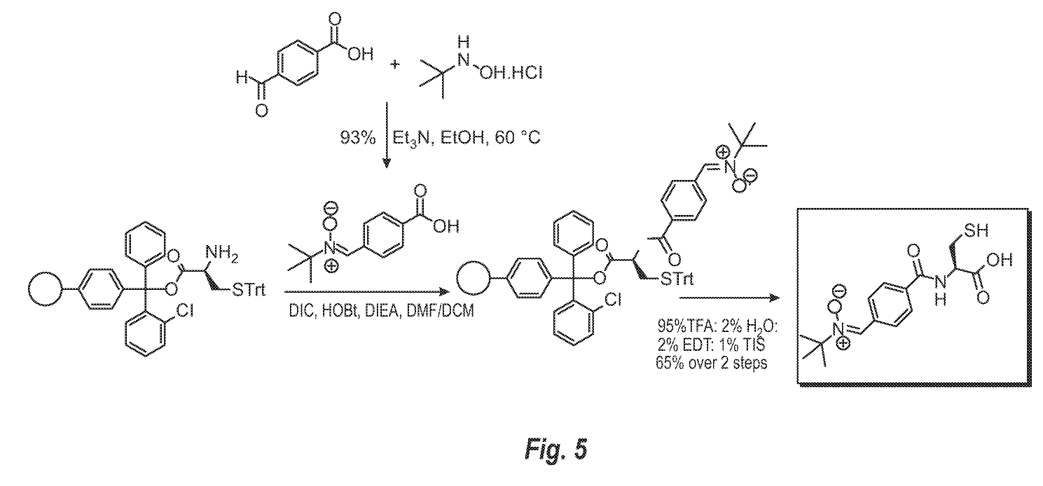

FIG. 4 illustrates another synthesis method for producing an antioxidant. As shown, the “R” group can be any aromatic ring or rings, heteroaromatic ring(s), polycyclic compounds or the like. R as shown in FIG. 5 can be “E” as described in connection to Formula 1.

FIG. 5 illustrates a synthesis method for producing an antioxidant. Analytical HPLC-MS indicated that the product shown was obtained. Purification by flash column chromatography over silica-gel using 90% CHCl3: 8% MeOH: 2% AcOH yielded the desired compound. 1H and 13C NMR data were obtained. HRMS (m/z): [M+H] calculated for C15H21N2O4S, 325.1222; found, 325.1214. IR (NaCl): 3408, 2511, 1701, 1641, 1541, 1234 cm-1.

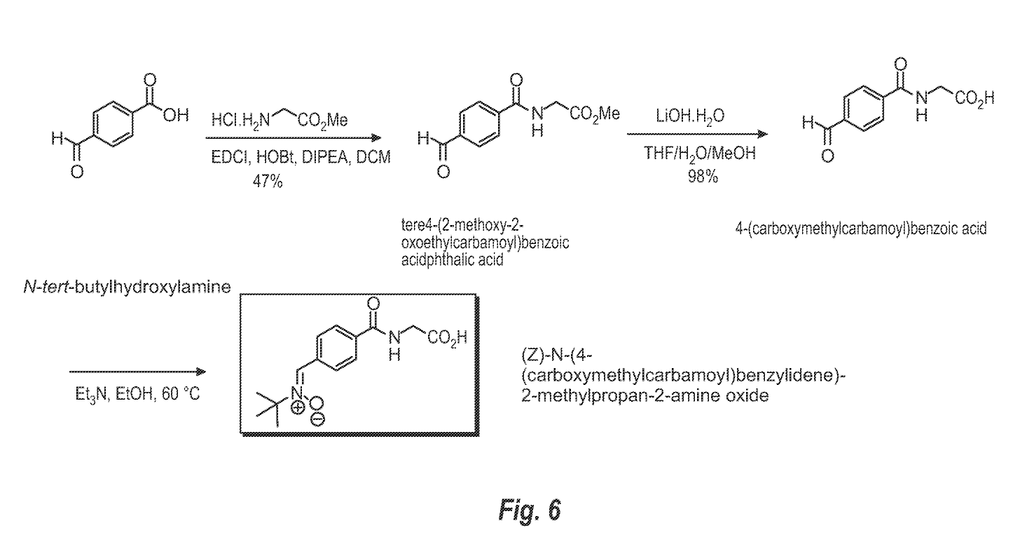

FIG. 6 illustrates a synthesis method for producing an antioxidant, (Z)-N-(4-(carboxymethylcarbamoyl)benzylidene)-2-methylpropan-2-amine oxide. NMR confirmed product. HRMS (m/z): [M+Na] calculated for C14H18N2O₄Na, 301.1164; found, 301.1188.

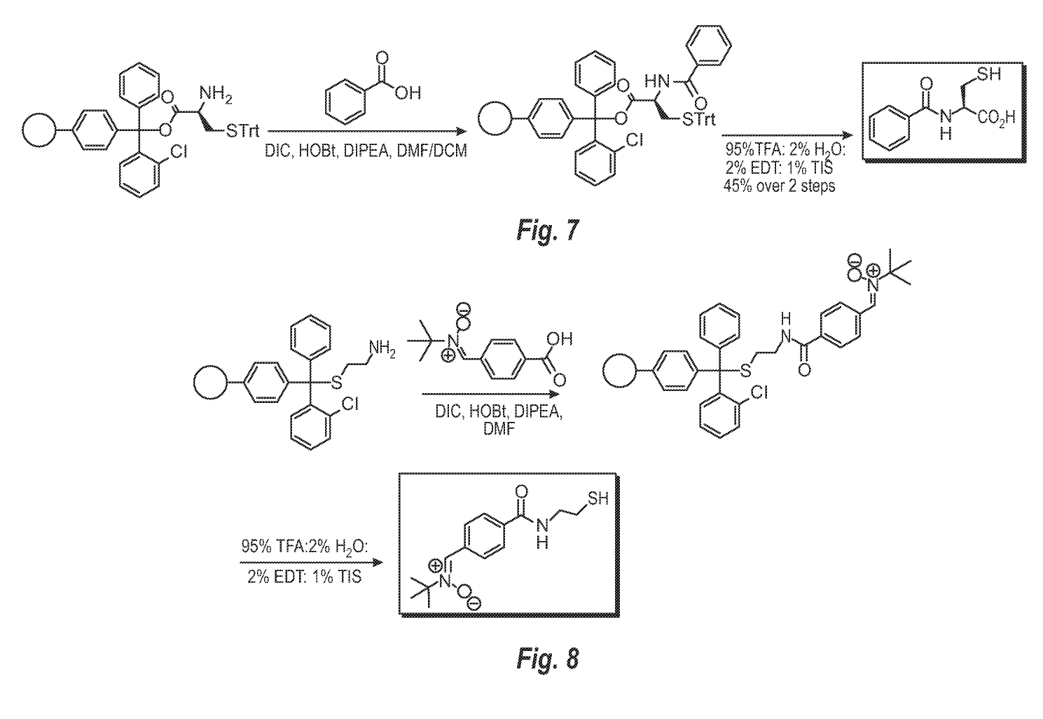

FIG. 7 illustrates a synthesis method for producing an antioxidant, R)-2-benzamido-3-mercaptopropanoic acid. NMR confirmed product. HRMS (m/z): [M+H] calculated for C10H12NO3S, 226.0538; found, 226.0540.

FIG. 8 illustrates a synthesis method for producing an antioxidant, (R,Z)-N-(4-(1-carboxy-2-mercaptoethylcarbamoyl)benzylidene)-2-methylpropan-2-amine oxide. NMR confirmed product. HRMS (m/z): [M+Na] calculated for C14H20N2O2SNa, 303.1143; found, 303.1140.

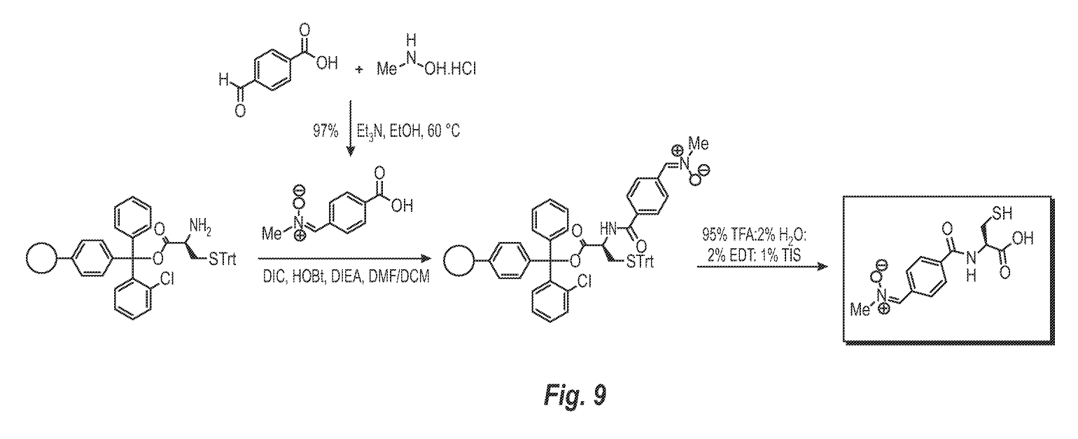

FIG. 9 illustrates a synthesis method for producing an antioxidant, (R,Z)-N-(4-(1-carboxy-2-mercaptoethylcarbamoyl)benzylidene)methanamine oxide. Synthesis of (Z)-N-(4-carboxybenzylidene)-2-methylpropan-2-amine oxide

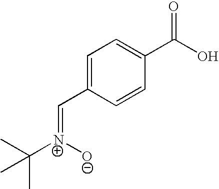

To a solution of 4-formylbenzoic acid (250 mg, 1.67 mmol) in ethanol (5 mL) were added triethylamine (0.28 mL, 2.00 mmol) and N-tert-butylhydroxylamine hydrochloride (251 mg, 2.00 mmol). The mixture was then heated at 60° C. for 4 hours. The solvent was evaporated in vacuo and the residue purified by flash column chromatography on silica gel using 1% acetic acid in ethyl acetate. The product was obtained as a white solid (320 mg, 1.56 mmol, 93.3%).

### Synthesis of (S,Z)-N-(4-(1-carboxy-2-mercaptoethylcarbamoyl)-benzylidene)-2-methylpropan-2-amine oxide

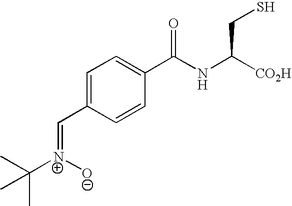

The H-Cysteine(trityl)-2-chlorotrityl resin (500 mg, 0.235 mmol, loading 0.47 mmol/g) is first swollen in DCM/DMF (10 mL, 1:1) for 2 hours in a manual peptide synthesis vessel. The solvent is then filtered off and a mixture of (Z)-N-(4-carboxybenzylidene)-2-methylpropan-2-amine oxide (242 mg, 1.18 mmol), diisopropylcarbodiimide (0.18 mL, 1.18 mmol), 1-hydroxybenzotriazole (159 mg, 1.18 mmol), and N,N′-diisopropylethylamine (0.41 mL, 2.35 mmol) in DMF (1 mL) were added to the resin suspended in DMF/DCM (5 mL, 1:1). A slow stream of nitrogen was bubbled through the synthesis vessel to agitate the resin beads. The progress of the coupling reaction was monitored by the Kaiser ninhydrin test which indicated that the coupling was complete after 4 hours. The reagent solution was then removed by vacuum filtration. The resin beads were thoroughly washed with DMF (3×5 mL), DCM (3×5 mL) and methanol (3×5 mL). The resin beads were then dried under vacuum for 3 hours.

The dried resin beads were then treated with a cleavage cocktail (5 mL) containing 95% TFA: 2% water: 2% ethanedithiol: 1% triisopropylsilane. Cleavage was allowed to proceed for 2 hours. The cleavage cocktail solution was then collected and evaporated in vacuo. Cold anhydrous diethyl ether was then added to the residue and an off-white precipitate was obtained. The ether solution was filtered off and the residue was triturated with cold ether (2×3 mL) and the crude precipitate was then dried under vacuum. The crude precipitate was purified by flash column chromatography on silica gel using 90% chloroform: 8% methanol: 2% acetic acid to yield 22 mg (65.2%) of an off-white semi-solid. ¹H NMR (400 MHz, MeOD) δ 8.54-8.33 (m, 2H), 8.11-7.84 (m, 3H), 4.77 (S, 1H), 3.64-3.40 (m, 1H), 3.21-2.94 (m, 1H), 1.72-1.45 (m, 9H). ¹³ C NMR (101 MHz, MeOD) δ 173.55, 167.88, 135.19, 133.65, 130.62, 129.32, 128.71, 72.85, 55.30, 28.52, 26.57. HRMS (m/z): [M+H] calculated for C₁₅H₂₁N₂O₄S, 325.1222; found, 325.1214.

### Synthesis of (Z)-N-(4-(2-mercaptoethylcarbamoyl)benzylidene)-2-methylpropan-2-amine oxide

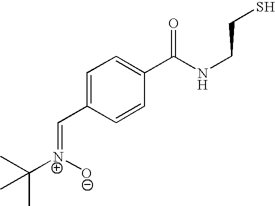

The procedure and purification was the same as that followed for the preparation of (S,Z)-N-(4-(1-carboxy-2-mercaptoethylcarbamoyl)-benzylidene)-2-methylpropan-2-amine oxide. The resin used in this case was 2-chlorotritylcysteamine (500 mg, 0.250 mmol, loading 0.5 mmol/g). The product was obtained as a colorless oil (16 mg, 23%). ¹H NMR (500 MHz, CDCl₃) δ 8.35 (d, J=8.5, 2H), 7.83 (d, J=8.5, 2H), 7.62 (s, 1H), 6.84 (br. s, 1H), 3.68-3.61 (m, 2H), 2.87-2.75 (m, 2H), 1.63 (s, 9H). ¹³C NMR (126 MHz, CDCl₃) δ 166.96, 135.11, 133.98, 129.33, 128.98, 127.25, 71.66, 43.04, 28.50, 24.83. HRMS (m/z): [M+Na] calculated for C₁₄H₂₀N₂O₂SNa, 303.1143; found, 303.1140.

### Synthesis of (Z)-N-(4-(carboxymethylcarbamoyl)benzylidene)-2-methylpropan-2-amine oxide

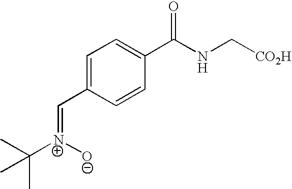

To a solution of (Z)-N-(4-carboxybenzylidene)-2-methylpropan-2-amine oxide (300 mg, 1.46 mmol) in dry DCM were added glycine methyl ester hydrochloride (202 mg, 1.61 mmol), EDCI (454 mg, 2.92 mmol), 1-hydroxybenzotriazole (197 mg, 1.4 mmol), and N,N′-diisopropylethylamine (0.51 mL, 2.92 mmol). The mixture was stirred at room temperature under argon overnight. The reaction mixture was then washed with saturated aqueous NaHCO₃ (3×10 mL), water (3×10 mL), and brine (3×10 mL). The organic layer was then dried over anhydrous Na₂SO₄ and the solvent evaporated in vacuo. Flash column chromatography on silica gel using 1% acetic acid in ethyl acetate yielded the product as a pale-yellow oil (214 mg, 0.782 mmol, 53.6%). ¹H NMR (500 MHz, CDCl₃) δ 11.70 (s, 1H), 8.31 (d, J=8.5, 2H), 7.87 (d, J=8.5, 2H), 7.64 (s, 1H), 7.35 (s, 1H), 4.15 (d, J=4.9, 2H), 1.62 (s, J=5.8, 9H). ¹³C NMR (126 MHz, CDCl₃) δ 173.33, 167.16, 134.91, 133.71, 130.64, 129.18, 127.57, 71.68, 45.99, 28.45. HRMS (m/z): [M+Na] calculated for C₁₄H₁₈N₂O₄Na, 301.1164; found, 301.1188.

### Synthesis of (S)-2-benzamido-3-mercaptopropanoic acid

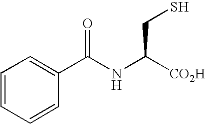

The procedure and purification was the same as that followed for the preparation of (S,Z)-N-(4-(1-carboxy-2-mercaptoethylcarbamoyl)-benzylidene)-2-methylpropan-2-amine oxide. In this case the resin (1.00 g, 0.470 mmol, loading 0.47 mmol/g) was coupled with benzoic acid (287 mg, 2.35 mmol). The product was obtained as a colorless oil (48 mg, 45.3%). ¹H NMR (400 MHz, CDCl₃) δ 9.37 (s, 1H), 7.82 (d, J=7.0, 2H), 7.56-7.36 (m, 3H), 5.06 (d, J=3.0, 1H), 3.24-3.06 (m, 2H). ¹³C NMR (101 MHz, CDCl₃) δ 173.34, 168.29, 133.30, 132.52, 128.97, 127.48, 54.35, 26.87. HRMS (m/z): [M+H] calculated. for C₁₀H₁₂NO₃S, 226.0538; found, 226.0540.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein by specific reference.

1. An antioxidant compound as in Formula 1, and its pharmaceutically acceptable salts, enantiomers, diastearomers, N-oxides, prodrugs or metabolites, or the like: 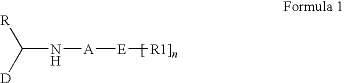
wherein one or more of R, R1, or D is an antioxidant substituent;
R and D are each independently selected from hydrogen or groups containing cysteine, thiols, disulfides, amino acids, sulfur, phosphate, amines, amides, or carboxylic acids;
when one of R or D is hydrogen, the other includes a thiol, disulfide, sulfur, phosphate, or carboxylic acid;
A can be CO, SO, SO₂,or C═S;
E is one or more ring groups which are aromatic, carbocyclic, and/or heterocyclic 5, 6 and 7 membered rings being mono, bi, tri, tetra, penta, hexa, hepta or octa cyclic fused rings that are substituted or unsubstituted, each heterocyclic ring can include one or more hetero atoms chosen from to O, S, N, Se, or P;
each R1 is at a para, meta, and/or ortho position;
n is 1, 2, 3, 4, or 5 for each ring; and
each R1 is a hydrogen, aliphatic, aromatic, or an antioxidant substituent.

2. An antioxidant as in claim 1, wherein the antioxidant substituents are selected from cysteines, nitrones, alkyliminyl oxides, oxaziridines, pyridine oxides, pyridine-1-oxides, piperidinyl-1-oxyls, isoindolines, methyl-oximidazolidine-1-oxyls, 3,5-di-tert-1-butyl-4-hydroxybenzamido groups, scorbates, derivatives thereof, or combinations thereof.

3. An antioxidant as in claim 1, wherein E is one or more of Benzene, Naphthalene, Anthracene, Phenanthrene, Benzopyrene, Coronene, Pyrene, Perylene, Triphenylene; Stilbene, Pyridine, Quinoline, Isoquinoline, Pyrazine, Quinoxaline, Acridine, Pyrimidine, Quinazoline, Pyridazine, Cinnoline, Furan, Benzofuran, Isobenzofuran, Pyrrole, Indole, Isoindole, Thiophene, Benzothiophene, Benzo[c]thiophene, Imidazole, Benzoimidazole, Purine, Pyrazole, Indazole, Oxazole, Benzoxazole, Isoxazole, Benzisoxazole, Thiazole, Benzothiazole, 2H-Pyrane, Azulene, isoalloxazine, 1,2,3-Triazine, 1,2,4-Triazine, 1,3,5-Triazine, 2,4,6-Triphenylphosphorine, or thiophene.

4. An antioxidant as in claim 1, when each R1 is independently selected from the group H, F, Cl, Br, I, —OH, —CF₃, —OR2, —CN, —NO₂, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, —C(NO)C(CH₃)₃, oxaziridine ring, nitrone or ozaziridine ring with C1-C6 straight or branched substituted or unsubstituted saturated or unsaturated alkyl, where p can be 0, 1, or 2, and R2 is an aromatic or an aliphatic group.

5. An antioxidant as in claim 4, when included R2, at each occurrence, is independently selected from the group H, OH, C1-C6 alkyl, C2-C6 alkenyl, C2-C6 alkynyl, 5-7 membered saturated or unsaturated aromatic or carbocycle that is substituted or unsubstituted, 5-7 membered saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C3-C14 saturated or unsaturated aromatic or carbocycle that is substituted or unsubstituted, —C(O)C3-14 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)O-5-7 membered saturated or unsaturated aromatic or carbocycle that is substituted or unsubstituted and with one or more heteroatoms.

6. An antioxidant as in claim 5, wherein the heteroatoms are nitrogen, oxygen, sulfur, or selenium.

7. An antioxidant as in claim 5, when substituted, R2 is substituted with R3, which at each occurrence, is independently selected from the group F, Cl, Br, OH, I, ═O, ═S, ═NR4, ═NOR4, ═N—NR4R4, —CF₃, —OR4, —CN, —NO₂, —NR4R4, —C(O)R4, —C(O)OR4, —OC(O)R4, —C(O)NR4R4, —NR4C(O)R4, —OC(O)NR4R4, —NR4C(O)OR4, —NR4C(O)NR4R4, —C(S)R4, —C(S)OR4, —OC(S)R4, —C(S)NR4R4, —NR4C(S)R4, —OC(S)NR4R4, —NR4C(S)OR4, —NR4C(S)NR4R4, —C(NR4)R4, —C(NR4)OR4, OC(NR4)R4, —C(NR4)R4R4, —NR4C(NR4)R4, —OC(NR4)R4, —NR4C(NR4)OR4, —NR4C(NR4)NR4R4, S(O)ₚR4, —SO₂NR4R4, —R4, —C(NO)C(CH₃)₃, oxaziridine ring, nitrone or ozaziridine ring with C1-C6 straight or branched substituted or unsubstituted saturated or unsaturated alkyl, wherein R4 is an aromatic or aliphatic group, substituted or unsubstituted.

8. An antioxidant as in claim 7, when included R4, at each occurrence, is independently selected from the group H, C1-C6 alkyl, C2-C6 alkenyl, C2-C6 alkynyl, 5-7 membered saturated or unsaturated aromatic or carbocycle that is substituted or unsubstituted, 5-7 membered member saturated or unsaturated aromatic or carbocycle that is substituted or unsubstituted and with one or more heteroatoms, —C(O)C1-C6 alkyl, —C(O)C2-C6 alkenyl, —C(O)C2-C6 alkynyl, —C(O)C5-C7 saturated or unsaturated aromatic or carbocycle that is substituted or unsubstituted, —C(O)C5-C7 saturated or unsaturated aromatic or carbocycle that is substituted or unsubstituted and with one or more heteroatoms, —C(O)O—C1-C6 alkyl, —C(O)O—C2-C6 alkenyl, —C(O)O—C2-C6 alkynyl, —C(O)OC5-C7 saturated or unsaturated aromatic or carbocycle that is substituted or unsubstituted, —C(O)OC5-C7 saturated or unsaturated aromatic or carbocycle that is substituted or unsubstituted and with one or more heteroatoms. The heteroatoms can be nitrogen, oxygen, sulfur, and selenium, or the like.

9. An antioxidant as in claim 1, wherein R or D can independently be hydrogen, F, Cl, Br, I, —OH, —CF₃, —CH₂SH, —(CH₂)ₘSH, —(C1-C6 alkyl)-thiol, —(C1-C6 alkyl)disulfide, —CH₂-disulfide, —(CH₂)ₘ-disulfide, C(O)OH, —(C1-C6 alkyl)-C(O)OH, —OC1-C6 alkyl, —SH, —SC1-C6 alkyl, —CN, —NO2, —NH2, —NHC1-C6 alkyl, —N(C1-C6 alkyl)₂, —C(O)C1-C6 alkyl, —OC(O)C1-C6 alkyl, —C(O)OC1-C6 alkyl, —C(O)NH₂, —C(O)NHC1-C6 alkyl, —C(O)N(C1-C6 alkyl)₂, —NHC(O)C1-C6 alkyl, —SO₂NH₂, —SO₂NHC1-C6 alkyl, —SO₂N(C1-C6 alkyl)₂, and —S(O)ₚC1-C6 alkyl, 4-carboxyphenyl, amino acid, amine, or amide, each alkyl being substituted or unsubstituted, wherein m is 1, 2, 3, 4, 5, 6, 7, 8, 9, or 10.

10. An antioxidant as in claim 1, wherein R1 includes a sulfanyl, substituted sulfanyl, aminosulfonyl, substituted aminosulfonyl, sulfonic acid, sulfonic acid ester (i.e., sulfonate), carbamoyl, substituted carbamoyl, amino, substituted amino, hydroxyl, dihydroxyphosphoryl, substituted dihydroxyphosphoryl, aminohydroxyphosphoryl, substituted aminohydroxyphosphoryl, carboxy, or substituted carboxy.

11. An antioxidant as in claim 1, wherein R1 is CH₂CH₂COL, where L is OCH2CH(OH)M, and M is 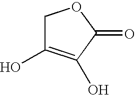
or E.

12. An antioxidant as in claim 11, wherein M is

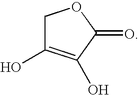

13. An antioxidant as in claim 1, wherein R1 is

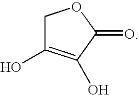

14. An antioxidant as in claim 1, wherein R1 is

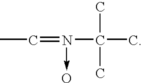

15. An antioxidant as in claim 1, when R1 is not an antioxidant each is independently selected from the group H, OH, F, Cl, Br, I, —CF₃, —OR2, —CN, —NO₂, NR2R2, —C(O)R2, —C(O)OR2, —OC(O)R2, —C(O)NR2R2, —NR2C(O)R2, —OC(O)NR2R2, —NR2C(O)OR2, —NR2C(O)NR2R2, —C(S)R2, —C(S)OR2, —OC(S)R2, —C(S)NR2R2, —NR2C(S)R2, —OC(S)NR2R2, —NR2C(S)OR2, —NR2C(S)NR2R2, —C(NR2)R2, —C(NR2)OR2, —OC(NR2)R2, —C(NR2)NR2R2, —NR2C(NR2)R2, —OC(NR2)NR2R2, —NR2C(NR2)OR2, —NR2C(NR2)NR2R2, —S(O)ₚR2, —SO₂NR2R2, R2, —C(NO)CH₃, —C(NO)C1-C6 alkyl, or the like. R2 can be an aliphatic or aromatic.

16. An antioxidant as in claim 1, wherein the antioxidant is a cysteine nitrone.

17. An antioxidant as in claim 1, wherein the antioxidant has a chemical structure of Formula 2: 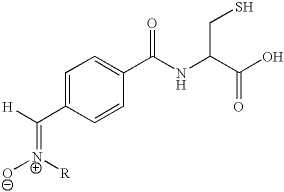
wherein the R group of Formula 2 includes at least one of an alkyl, ring, alkyliminyl oxide, aliphatic, aromatic, and/or aryl group.

18. An antioxidant as in claim 17, wherein the alkyl or aliphatic can be straight, branched, substituted, unsubstituted, saturated, unsaturated, and where the ring, aromatic, or aryl group can be fused, linked, homoatom, heteroatom, substituted, unsubstituted, saturated, unsaturated, or the like.

19. An antioxidant as in claim 1, wherein the antioxidant is a cysteine nitrone compound having Formula 3, Formula 4, Formula 5, Formula 6, Formula 7: 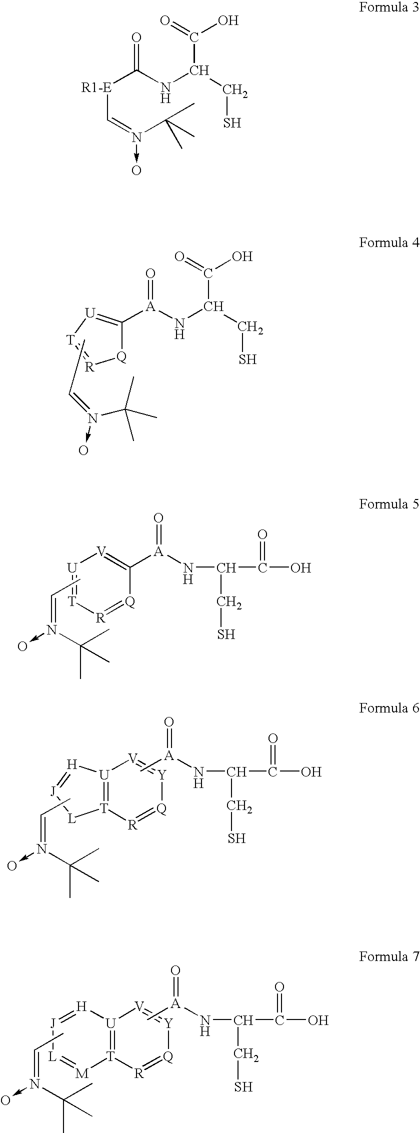
wherein in Formula 3, Formula 4, Formula 5, Formula 6, or Formula 7, the substituents H, J, L, M, Q, R, T, U, V are independently selected from CH₂, O, NH, S, CH, and N; and
A is C or S.

20. An antioxidant as in claim 1, wherein the antioxidant is selected from:
2-[4-(tert-Butylnitrone)-benzoylamino]-3-mercapto-propionic acid;
(R,Z)-N-(4-(1-carboxy-2-mercaptoethylcarbamoyl)benzylidene)-2-methylpropan-2-amine oxide;
(Z)-N-(4-(carboxymethylcarbamoyl)benzylidene)-2-methylpropan-2-amine oxide;
(R)-2-benzamido-3-mercaptopropanoic acid;
(R,Z)-N-(4-(1-carboxy-2-mercaptoethylcarbamoyl)benzylidene)methanamine oxide;
(2R)-2-(4-(2-tert-butyl-1,2-oxaziridin-3-yl)benzamido)-3-mercaptopropanoic acid;
(R,Z)-N-(4-(1-carboxy-2-mercaptoethylcarbamoyl)benzylidene)-2-methylpropan-2-amine oxide;
4-(4-(1-carboxy-2-mercaptoethylcarbamoyl)phenyl)pyridine-1-oxide;
2-(3,5-di-tert-butyl-4-hydroxybenzamido)-3-mercaptopropanoic acid;
(R,Z)-N-4-(1-(4-carboxyphyenyl)-2-mercaptoethylcarbamoyl)benzylidene)-2-methylpropan-2-amine oxide;
2-[2-(tert-Butylnitrone)-benzoylamino]-3-mercapto-propionic acid;
2-{[6-(tert-Butylnitrone)-pyridine-3-carbonyl]-amino}-3-mercapto-propionic acid;
2-{[5-(tert-Butylnitrone)-pyridine-2-carbonyl]-amino}-3-mercapto-propionic acid;
2-{[2-(tert-Butylnitrone)-pyrimidine-5-carbonyl]-amino}-3-mercapto-propionic acid;
2-{[6-(tert-Butylnitrone)-pyridazine-3-carbonyl]-amino}-3-mercapto-propionic acid;
2-{[5-(tert-Butylnitrone)-thiophene-2-carbonyl]-amino}-3-mercapto-propionic acid;
2-{[4-(tert-Butylnitrone)-thiophene-2-carbonyl]-amino}-3-mercapto-propionic acid;
2-{[5-(tert-Butylnitrone)-furan-2-carbonyl]-amino}-3-mercapto-propionic acid;
2-{[5-(tert-Butylnitrone)-1-H-pyrrole-2-carbonyl]-amino}-3-mercapto-propionic acid;
2-{[5-(tert-Butylnitrone)-1-methyl-pyrrole-2-carbonyl]-amino}-3-mercapto-propionic acid;
2-{[8-(tert-Butylnitrone)-2,4-dioxo-2,3,4,10-tetrahydro-benzo[g]pteridine-6-carbonyl]-amino}-3-mercapto-propionic acid;
2-{[7-(tert-Butylnitrone)-naphthalene-2-carbonyl]-amino}-3-mercapto-propionic acid;
2-{[7-(tert-Butylnitrone)-anthracene-2-carbonyl]-amino}-3-mercapto-prop ionic acid;
2-{[5-(tert-Butylnitrone)-pyridine-3-carbonyl]-amino}-3-mercapto-propionic acid;
2-{[3-(tert-Butylnitrone)-quinoline-6-carbonyl]-amino}-3-mercapto-propionic acid;
2-{[6-(tert-Butylnitrone)-quinoline-3-carbonyl]-amino}-3-mercapto-propionic acid;
2-{[3-(tert-Butylnitrone)-isoquinoline-6-carbonyl]-amino}-3-mercapto-propionic acid;
2-{[6-(tert-Butylnitrone)-isoquinoline-3-carbonyl]-amino}-3-mercapto-propionic acid;
2-{[6-(tert-Butylnitrone)-pyrazine-2-carbonyl]-amino}-3-mercapto-propionic acid;
2-{[7-(tert-Butylnitrone)-quinoxaline-2-carbonyl]-amino}-3-mercapto-propionic acid;
2-{[7-(tert-Butylnitrone)-acridine-2-carbonyl]-amino}-3-mercapto-propionic acid;
2-{[5-(tert-Butylnitrone)-pyridazine-3-carbonyl]-amino}-3-mercapto-propionic acid;
2-{[4-(tert-Butylnitrone)-pyrimidine-2-carbonyl]-amino}-3-mercapto-propionic acid;
2-{[7-(tert-Butylnitrone)-quinazoline-2-carbonyl]-amino}-3-mercapto-propionic acid;
2-{[6-(tert-Butylnitrone)-cinnoline-3-carbonyl]-amino}-3-mercapto-propionic acid;
2-{[2-(tert-Butylnitrone)-7aH-indene-5-carbonyl]-amino}-3-mercapto-propionic acid;
2-{[2-(tert-Butylnitrone)-2H-indole-5-carbonyl]-amino}-3-mercapto-propionic acid;
2-{[3-(tert-Butylnitrone)-3aH-isoindole-5-carbonyl]-amino}-3-mercapto-propionic acid;
2-{[2-(tert-Butylnitrone)-1H-indole-5-carbonyl]-amino}-3-mercapto-propionic acid;
2-{[2-(tert-Butylnitrone)-benzo[b]thiophene-5-carbonyl]-amino}-3-mercapto-propionic acid;
2-{[1-(tert-Butylnitrone)-benzo[c]thiophene-5-carbonyl]-amino}-3-mercapto-propionic acid;
2-{[2-(tert-Butylnitrone)-2H-benzoimidazole-5-carbonyl]-amino}-3-mercapto-1-propionic acid;
2-{[8-(tert-Butylnitrone)-8H-purine-2-carbonyl]-amino}-3-mercapto-prop ionic acid;
2-{[2-(tert-Butylnitrone)-benzooxazole-5-carbonyl]-amino}-3-mercapto-propionic acid;
2-{[3-(tert-Butylnitrone)-2H-indazole-6-carbonyl]-amino}-3-mercapto-propionic acid;
2-{[3-(tert-Butylnitrone)-benzo[d]isoxazole-6-carbonyl]-amino}-3-mercapto-propionic acid;
2-{[3-(tert-Butylnitrone)-benzo[d]isothiazole-6-carbonyl]-amino}-3-mercapto-propionic acid;
2-{[5-(tert-Butylnitrone)-[1,2,4]triazine-3-carbonyl]-amino}-3-mercapto-propionic acid;
2-{[6-(tert-Butylnitrone)-[1,2,3]triazine-4-carbonyl]-amino}-3-mercapto-propionic acid;
2-{[4-(tert-Butylnitrone)-[1,3,5]triazine-2-carbonyl]-amino}-3-mercapto-propionic acid; or
2-{[7-(tert-Butylnitrone)-pyrene-1-carbonyl]-amino}-3-mercapto-propionic acid.

21. An antioxidant as in claim 1, wherein the antioxidant is present in a therapeutically effective amount in a subject.

22. An antioxidant as in claim 21, wherein the subject has a condition that can be treated, inhibited, and/or prevented with antioxidant therapy.

23. An antioxidant as in claim 22, wherein the subject has the antioxidant in a therapeutically effective amount for:
treating, inhibiting, and/or preventing bronchopulmonary disease, asthma, diabetes mellitus, myocardial infarction, damage caused by drug abuse and/or overdose, acetaminophen toxicity, alcoholism, burn injury, acute radiation exposure, Alzheimer's disease, Parkinson's disease, cerebral ischemia, cerebral stroke, traumatic brain injury, acute spinal cord injury, alopecia, aging, inflammatory bowel disease, autoimmune disorders, preterm parturition, premature cervical ripening, pregnancy loss, cerebrovascular stroke, retinal ischemia, macular degeneration, degenerative disorders of the retina, renal ischemia, arteriosclerosis, cardiovascular diseases, amyotrophic lateral sclerosis, Huntington's disease, multiple sclerosis, head trauma, nerve injury, neuropathies, migraine, schizophrenia, mood disorders, pancreatitis, pancreatic disorders, diabetes, epilepsy, transplant and graft failure or rejection, hepatitis, jaundice-induced liver disorders, lung injury or damage, gastric ulcer, endotoxemia, aging or senescence, preterm labor, fetal damage due to intrauterine ischemia, pain syndromes acute and chronic or neuropathic, arthritis, autoimmune disorders, asthma, allergic reactions, inflammatory bowel disease, irritable bowel syndrome, uveitis, cancer, complications and disorders arising from cancer therapy, alopecia, fetal inflammatory syndrome, arthritis, uveitis, obesity, eating disorders, cancer, sleeping disorders, cognition, depression, anxiety, high blood pressure, lipid disorders and atherosclerosis, abnormal intrauterine conditions; newborn brain abnormalities; intrauterine oxygen deficiency, either chronic or acute; perinatal brain damage; acute oxygen depravation; perinatal hypoxic ischemic reperfusion injury; brain damage associated with acute oxygen depravation; conditions associated with primary cell death due to lack of oxygen; conditions associated with secondary cell death due to inflammatory response by reperfusion with oxygenated blood; acute ischemic reperfusion during labor; cerebral palsy; intrauterine growth restriction (IUGR); chronic fetal hypoxemia; fetal inflammatory response syndrome and its multiorgan sequelae including the brain; preterm labor and/or birth associated with fetal inflammatory response; idiopathic preterm birth; or other condition caused by excessive oxidation, or combinations thereof; or
modifying mammalian inflammatory pathways.

24. An antioxidant as in claim 1, wherein the antioxidant is present in a pharmaceutical composition.

25. An antioxidant as in claim 24, wherein the antioxidant is present in the pharmaceutical composition in a therapeutically effective amount.

26. An antioxidant as in claim 25, the pharmaceutical composition further includes a pharmaceutically acceptable carrier.
